(19) United States
(12) Patent Application Publication
(10) Pub. No.: US 2004/0022779 A1
(43) Pub. Date: Feb. 5, 2004

(54) Method for identifying apoptosis-modified proteins

(76) Inventors: Thomas Rudel, Berlin (DE); Bernd Thiede, Dublin (IE); Nikolaus Machuy, Berlin (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/297,619

(22) PCT Filed: Jun. 15, 2001
(86) PCT No.: PCT/EP01/06780

(30) Foreign Application Priority Data
Jun. 16, 2000 (EP) 00112813.1
Nov. 16, 2000 (EP) 00125013.3

(51) Int. Cl. G01N 33/574; A61K 38/48; C12N 9/64

(52) U.S. Cl. 424/94.63; 435/7.23; 435/226

(57) ABSTRACT

The present invention relates to a method for characterizing or identifying apoptosis-modified proteins which are expressed by cells, preferably human cells. Further, novel apoptosis-modified proteins are provided which are suitable as targets for diagnosis, prevention or treatment of diseases, particularly hyperproliferative or degenerative diseases.

The present invention relates to a method for characterizing and/or identifying apoptosis-modified proteins which are expressed by cells, preferably mammalian cells, more preferably T-cells, most preferably human T-cells. Further, novel apoptosis-modified proteins are provided which are suitable as targets for diagnosis, prevention or treatment of diseases, particularly hyperproliferative or degenerative diseases. The invention also relates to the modification of caspase cleavage sites in proteins to prevent their cleavage by caspases, to the use of caspase cleavage sites to screen for or design substances that are able to block cleavage as well as use of caspase cleavage site containing proteins as diagnostic tools for detecting caspase activity and/or inhibition of caspase activity.

Apoptosis is an essential and complex process for the development and homeostasis of multicellular organisms. Improper regulation of this process results in various diseases including cancer, autoimmune disorders, viral infections, neurodegenerative disorders and myocardial infarction (1). The therapeutic regulation of apoptosis therefore offers numerous challenges (2).

Several components of the apoptotic cell death machinery were already identified. The best known contributors are the caspases (3,4) and their inhibitors (5) and substrates (6), the bcl-2 family (7,8), the death receptors (9), the mitochondria (10,11) and signal transduction pathways (12,13). Death receptors belong to the tumour necrosis factor (TNF) superfamily. The best characterized death receptors are Fas, TNFR1, DR3, DR4 and DR5. These receptors induce apoptosis by ligand binding and receptor oligomerization, recruitment of an adaptor protein to the death domain of the receptor. The adaptor molecule binds a caspase, thereby activating the apoptosis machinery. On the other hand decoy receptors compete with specific death receptors for ligand binding.

However, hundreds of stimuli induce apoptosis independent of death-receptor like UV or γ-irradiation, chemotherapeutic drugs and viral or bacterial infections. The apoptotic phenotype is very similar in all apoptotic cells independent of the stimuli used to induce apoptosis. In addition, apoptosis of cells from organisms which are evolutionary distantly related, like nematodes and man, is regulated by structurally related proteins like caspases and these cells show similar phenotypes. These findings together were the basis for a concept of a highly conserved apoptotic machinery involving similar factors in all cells.

The Fas receptor (CD95 or Apo1) plays an important role in immune regulation by deletion of autoimmune cells and activation-induced T-cell death, killing of targets such as virus-infected cells or cancer cells by cytotoxic T-cells and by natural killer cells and killing of inflammatory cells at immune privileged sites (14-16). Fas is expressed in a wide variety of cells, whereas the Fas ligand (FasL) has a limited tissue distribution. FasL is rapidly induced in activated T-cells and natural killer cells but few other cells appear to express significant levels of FasL. The decoy receptor DcR3 binds to FasL and inhibits FasL-induced apoptosis (17). Thus, tumours may be able to evade the death signal by binding of a trigger of apoptosis. Cis-platin causes intra-DNA strand cross links. DNA damage induced by cis-platin ultimately induces apoptosis in a variety of cell lines.

Proteome approaches have been used to find new apoptosis-associated proteins (18). However, the conditions used in these studies to induce apoptosis allowed synthesis of new proteins because (1) protein synthesis was not blocked by the addition of protein synthesis inhibitors such as cycloheximide and (2) the cells were stimulated to undergo apoptosis for such a long time (more than 12 h) that synthesis of new proteins was possible. The modified proteins obtained by this treatment thus consisted of apoptosis-modified proteins and proteins which were expressed as a general response of the cell to stress. The identification of a protein as apoptosis-modified was thus not possible.

Thus, the object underlying the present invention was to provide a method allowing characterization or identification of apoptosis-modified proteins, which does not suffer from the disadvantages as described above.

In order to solve this problem we induced apoptosis by the addition of Anti-Fas IgM antibody in a defined way for 6 h or cis-platin for 16 h and at the same time blocked the synthesis of new proteins by the addition of cycloheximide. Under these conditions only apoptosis-modified proteins, and not newly synthesised proteins, were detected. This is also very important for the apoptosis-induced translocation of proteins which can be attributed to the movement of a pre-formed protein upon apoptosis induction. Translocation from the cytosol to the nucleus in apoptotic cells of the pre-formed caspase activated DNAse (CAD) is shown in (33).

Translocation of Bid from the cytosol to the mitochondria is the critical event in Fas-induced apoptosis in several cell lines. Thus interference with apoptosis-induced translocation of proteins might be of therapeutic use to either trigger apoptosis in proliferative diseases or to prevent apoptosis in degenerative diseases.

Thus, the present invention provides a proteome analysis of cells to characterize and/or identify apoptosis-associated and particularly apoptosis-modified proteins. Subtractive analysis of two dimensional gel-electrophoresis patterns of apoptotic cells and non-apoptotic cells revealed differences in a plurality of protein spots. The predominantly altered protein spots were identified after proteolytic digestion and peptide mass fingerprinting. Of the identified proteins, the heterogeneous nucleoprotein (hnRNP) A/B, hnRNP A2/B1, hnRNP A3, hnRNP D, hnRNP F, hnRNP H, hnRNP I, hnRNP K, hnRNP L, hnRNP R, hnRNP JKTBP1, hnRNP A0, and Apobec-1 interacting protein, the splicing factors SRp30c, P54nrb, SF2p33 (ASF-2), SF SC35, NMP200 (related to SR PRP19) and PTB-associated SF, splicing factor 1, and KH-type splicing regulatory protein, the translation factors EF-Tu, EF-1 beta, EIF-5A, 40 S ribosomal protein SA, elongation factor 1-delta, elongation initiation factor 3 (subunit 4) and poly(A)-binding protein (cytoplasmic 4), the structural proteins gamma-actin and the myosin heavy chain, the factors involved in signal transduction GAP SH3 binding protein, cGMP-dependent protein kinase, GAP SH3 protein 2, and the small G protein, the chromatins type I alpha, Baf-57, CAF-1 (RB b.p.) (WD-repeats) and KIAA1470, the transcription factor CBF-beta, the proteasomal factor 26S protease SU 12, proteasome subunit C8 and Tat binding protein-1, the mitchondrial factors isocitrate dehydrogenase, AOP-1, ATP synthase beta chain and ATP synthase D chain and the diverse factors SYT interacting protein SIP, PA1-G, CRHSP-24, HCD2, GMP synthase, FUSE binding protein 1, HDGF, alpha NAC, ARDH, cargo selection protein, DAZ associated protein 1, DEAD box protein retinoblastoma, dihydrofolate reductase, hydroxyacyl-CoA dehydrogenase/3-ketoacyl-CoA thiolase/enoyl-CoA hydratase, ER-60, HCA56, Hsp-105, IGF-II mRNA-binding protein 1, IGF-II mRNA-binding protein 3, lactate dehydrogenase A, NS-associated protein, RAD 21, RAD 23 homolog B, T-complex protein 1 beta subunit, thioredoxin like protein, an unnamed protein (NCBI 7020309), chondrosarcoma-associated protein 2, ELAV-like 1 (Hu antigen R), HnRNP M, HnRNP E1, SKI interacting protein, glutathione S-transferase, VDAC 3, mortalin-2 (heat shock 70 kd protein 9B), prohibitin, 26S protease regulatory subunit 4, and proteasome subunit alpha type 1 were hitherto unknown to be involved in apoptosis. HnRNP C1/C2, nucleolin, p54nrb, Rho GDI2, ASF-2, SRp30c and BTF3 include aspartic acid/glutamic acid-rich domains and hn RNP A2/B1, hn RNP C1/C2, nucleolin and BTF3 interact with protein kinase CK2. Remarkably, the heterogeneous nuclear ribonucleoprotein (hnRNP) A/B, hnRNPs A1, hnRNP A2/B1, hnRNP A3, hnRNP C1/C2, hnRNP I, hnRNP F, hnRNP H, hnRNP 1, hnRNP K, hnRNP L, hnRNP R, hnRNP JKTB1, the splicing factors SRp30c, P54nrb, SF2p33 (ASF-2), SFSC35, PTB-associated SF, the signal transduction protein GAP SH3 binding protein, the chromatin associated protein nucleolin, hnRNP A0, Apobec-1 interacting protein, elongation initiation factor 3 (subunit4), poly(A)-binding protein (cytoplasmic4), GAP SH3-binding protein 2, DAZ associated protein 1, IGF-II mRNA-binding protein 1, IGF-II mRNA-binding protein 3, NS-associated protein, Hn RNP M and ELAV-like 1 contain the RNP motif. The proteins splicing factor 1, KH-type splicing regulatory protein, IGF-II mRNA-binding protein 1, IGF-II mRNA-binding protein 3 and Hn RNP E1 contain the KH motif. Prohibitin is known to be an inhibitor of DNA synthesis, Hsp-60 and Mortalin-2 are known to be chaperones. VDAC 3 is known to be an ion channel. The proteins PFC6D, KNFE3 (partial sequence TPGT(F/Mox)E) and KPF1 were unknown.

Particularly preferred apoptosis-modified proteins are GAP SH3 binding protein, HCD2 and AOP-1.

‘Modification’ or ‘apoptosis-modified’ in this context describes the alteration of a protein in a given compartment during the process of apoptosis. The protein spot elicits changes in the size or the charge or the size and the charge. These changes may be due to transcriptional (e.g. splicing), translational and/or posttranslational (e.g. glycosylation and/or proteolyis) variations. Furthermore, the protein may be translocated. ‘Translocation’ in this context describes differences in the localisation of a protein in compartments of apoptotic cells compared to the compartments of non-apoptotic cells.

The method established and described above can be used for other cell types expressing death receptors like TNF-receptor, DR-3, DR-4 or DR-5 or any receptor which induces apoptosis in the absence of protein biosynthesis. The method can be used for cells induced to undergo apoptosis by other pathways than the receptors described above.

Thus, a subject matter of the present invention is a method for characterizing and/or identifying apoptosis-modified proteins comprising the steps:

(a) providing a first extract and a second extract comprising soluble proteins, wherein said first extract is from a cell without apoptosis induction and said second extract is from a cell after apoptosis induction,

(b) separating said first and second extracts by two-dimensional gel electrophoresis, wherein said first and second proteome patterns each comprising a plurality of protein species are obtained

(c) comparing said first and second proteome patterns and

(d) characterizing and/or identifiying apoptosis-modified protein species.

In the context of the present application, characterization of a protein is the analysis of the chemical composition of the protein. Identification of a protein is the assignment of a spot on the 2-DE gel to its biological functions or at least the assignment to a gene including the regulatory encoding sequences. In the context of the present invention, the proteome comprises the protein composition of a cell or a part of it at a defined biological situation (19).

The method of the present invention allows characterization and identification of apoptosis-modified proteins from cells, preferably from mammalian cells, more preferably from human cells, such as mammalian and particularly human T-cells, e.g. from an immortalized T-cell line such as the T-cell line Jurkat E6 (ATCC TIB 152).

Step (a) of the method of the invention comprises the preparation of extracts comprising soluble proteins. A first extract is obtained from a cell without apoptosis induction and a second extract is obtained from a cell after apoptosis induction. The extracts may be whole cell extracts but may be also extracts from cell compartments such as membranes, cytosol, mitochondria or nucleus. Apoptosis may be induced by contacting the cells with caspase activators and/or ligands of death receptors (such as an anti-Fas antibody) and/or cis-platin.

Preferably, the second extract is obtained from a cell wherein after apoptosis induction substantially no synthesis of new proteins has been allowed. This may be effected by adding an inhibitor of protein biosynthesis such as cycloheximide and/or by carrying out apoptosis induction for a period of time which is too short to allow a substantial synthesis of new proteins, e.g. a period of time of less than 12 h, preferably less than 8 h, e.g. about 6 h.

Step (b) of the method of the invention is a two-dimensional gel electrophoresis which comprises (i) separation in a first dimension according to the isoelectric point and (ii) separation in a second dimension according to size. The gel matrix is preferably a polyacrylamide gel. Gel preparation may be carried out according to known methods (20,21).

Step (c) of the method of the invention comprises comparing said first and second proteome patterns. This comparison may comprise a subtractive analysis of the first and second proteome patterns (22). By means of this subtractive analysis apoptosis-modified protein species are obtained which may be selected from protein species which (i) are located at different positions on the two-dimensional gels from the first and second extracts and/or (ii) have a different intensity on the two-dimensional gels from the first and second extracts.

The characterization of apoptosis-modified protein species may be carried out by peptide fingerprinting, wherein peptide fragments of the protein to be analysed are generated by in-gel proteolytic digestion, e.g. by digestion with trypsin. Further characterization of the peptides may be carried out by mass spectrometry, e.g. electrospray ionization mass spectrometry (ESI-MS) (23) and matrix-assisted laser dissorption/ionization mass spectrometry (MALDI-MS) (24) and/or by at least partial amino acid sequencing, e.g. by Edman degradation.

In a preferred embodiment, the invention further comprises as step (e) the determination if the apoptosis-associated modifications of the protein species are present in subjects, e.g. experimental animals or human patients suffering from apoptosis-associated diseases including hyperproliferative or degenerative diseases such as cancer, autoimmune and neurodegenerative disorders such as Alzheimer's disease, viral infections such as AIDS and vascular diseases such as myocardial infarction. By screening the presence of apoptosis-modified proteins in the patients, valuable targets for preventing or treating the above diseases may be identified.

A further subject matter of the present invention are proteomes from an apoptotic T-cell or a compartment thereof consisting of a pattern of individual proteins obtainable by the method as described above. The proteins consist of highly resolved patterns of proteins, comprising preferably at least 100, more preferably at least 500 and most preferably at least 1.000 different protein species, which are expressed by apoptotic T-cells. The term “protein species” describes a chemically clearly defined molecule in correspondence to one spot on a high performance 2-DE pattern. Preferably, the proteomes of the present invention, which may be in the form of two-dimensional gel electrophoresis pictures or electronic data bases thereof (25,26,27), contain the proteins as shown in Table 1 or at least a part thereof.

A still further subject matter of the present invention are individual proteins which are expressed by apoptotic cells, e.g. by apoptotic T-cells, and which have been characterized and identified by the method as described above. Preferably, these proteins are selected from heterogenous nuclear ribonucleoproteins such as hnRNP A/B (Gene bank Accession Number NM_{—}004499), A1 (X12671), A2/B1 (D28877), A3 (AF148457), C1/C2 (NM_{—}004500), D (D55671), F (L28010), H (L22009), I (NM_{—}002819), K (NM_{—}002140), L (NM_{—}001533), R (AF000364), JKTBP1 (D89092), hnRNP A0 (NM_{—}006805) and Apobec-1 interacting protein (U76713), splicing factors such as SRp30c (NM_{—}003769), p54nrb (U89867), SF2p33 (ASF-2) (M72709), SFSC35 (X62447), NMP200 (AJ131186), PTB-associated SF (NM_{—}05066), splicing factor 1 (Y08766), and KH-type splicing regulatory protein (NM_{—}003685), translational factors such as 60S acidic ribosomal protein (NM_{—}001002), EF-Tu (NM_{—}003321), EF-1β (NM_{—}001959), EIF-5A (NM_{—}001970), 40 S ribosomal protein SA (NM_{—}002295), elongation factor 1-delta (NM_{—}001960), elongation initiation factor 3 (subunit 4, AF020833), and poly(A-)binding protein (cytoplasmic 4, NM_{—}003819), structural proteins such as lamin B1 (L37747), lamin B2 (M94362), vimentin (NM_{—}003380) and beta-tubulin (V00599), the structural proteins gamma actin (M19283) and the myosin heavy chain (M31013), signal transduction proteins such as GAP SH3 binding protein (NM_{—}005754), Rho GDI2 (X69549), cGMP-dependent protein kinase type Iα (Z92867), GAP SH3 protein 2 (AF051311), and the small G protein (NM_{—}002872), chromatin associated proteins such as nucleolin (NM_{—}005381), Baf-57 (NM_{—}003079), CAF-1 (X71810), and KIAA 1470 (AB040903), transcription factors such as BTF3 (X53281) and CBF-β (L20298), proteasome subunits such as 26S protease subunit12 (NM_{—}002811), proteasome subunit C8 (NM_{—}002788) and Tat binding protein-1 (NM_{—}02804), mitochondrial proteins such as isocitrate dehydrogenase (NM_{—}002168), AOP-1 (NM_{—}006793), ATP synthase beta chain (M27132), ATP synthase D chain (NM_{—}006356), nucleophosmin (X16934), SYT interacting protein SIP (NM_{—}006328), PA1-G (NM_{—}002573), CRHSP-24 (AF115345), HCD2 (NM_{—}004493), GMP synthase (NM_{—}003875), FUSE binding protein 1 (NM_{—}003902), HDGF (NM_{—}004494), alpha NAC (NM_{—}005594), ARDH (X77588), cargo selection protein (NM_{—}005817), DAZ associated protein 1 (NM_{—}018959), DEAD box protein retinoblastoma (NM_{—}004939), dihydrofolate reductase (NM_{—}000791), hydroxylacyl-CoA dehydrogenase/3-ketoacyl-CoA thiolase/enoyl-CoA hydratase (NM_{—}000182), ER-60 (NM_{—}005313), HCA56 (AF220417), Hsp-105 (NM_{—}006644), IGF-II mRNA-binding protein 1 (NM_{—}006546), IGF-II mRNA-binding protein 3 (AF117108), lactate deydrogenase A (NM_{—}005566), NS-associated protein (NM_{—}006372), RAD 21 (X98294), RAD 23 homolog B (NM_{—}002874), T-complex protein 1 beta subunit (U91327), thioredoxin like protein (NM_{—}004786), an unnamed protein (NCBI 7020309, AK000310), c-Abl (P00519, pl 8.8, MW 140 kDa, determined by 2 DE gel electrophoresis), alpha-fodrin, Hsp-60, chondrosarcoma-associated protein 2, ELAV-like 1 (Hu antigen R), HnRNP M, HnRNP E1, SKI interacting protein, glutathione S-transferase, VDAC 3, mortalin-2 (heat shock 70 kd protein 9B), prohibitin, 26S protease regulatory subunit 4, and proteasome subunit alpha type 1. More preferably, these proteins are selected from the proteins as shown in Table 1 and the new proteins PFC6D, KNFE3 (partial sequence TPGT(F/Mox)E) and KPF1.

A still further subject matter of the present invention are proteins translocated from one cellular compartement such as nucleus, cytosol, mitochondria or membrane to another. Preferably, these proteins are selected from the protein species as described in Tables 3, 4, 5, 6, 7 or 8.

Especially preferred are apoptosis-associated and/or -modified proteins selected from GAP SH3 binding protein, HCD2 and AOP-1.

In addition to the proteins as specified above or fragments thereof having a length of preferably at least 10, more preferably at least 20 and most preferably at least 30 amino acids, the invention also relates to nucleic acids, e.g. DNA, RNA or nucleic acid analogs, e.g. DNA which encode these proteins or protein fragments or variants, e.g. allelic variants thereof. Further, the invention relates to substances capable of modulating the characteristics of the proteins or nucleic acids, e.g. antibodies, low molecular weight inhibitors or activators, antisense molecules or ribozymes.

The proteins or protein patterns as described above may be used as targets for the diagnosis, prevention or treatment of apoptosis-associated diseases or in a method for identifying apoptosis-modulators. A diagnostic method may comprise a determination of the presence or absence of apoptosis-modified proteins in a sample. A preventive or therapeutic method may comprise the activation or inhibition of apoptosis-modified proteins, e.g. an activation by overexpression via gene transfer into cells or organs by gene transfer vectors such as viruses, an inhibition by antisense or ribozyme molecules or an activation or inhibition by substances which modulate the amount, processing, presentation or conformation of the protein. The method for identifying apoptosis modulators (activators or inhibitors) may comprise a screening assay, e.g. a cellular or molecular screening assay which may be carried out in a high-throughput format.

Apoptosis modulators which are identified by the method of the present invention or compounds derived therefrom, e.g. by empirical derivatization and/or by computer modelling, may be provided as pharmaceutical compositions optionally together with suitable pharmaceutically acceptable carriers, diluents and/or adjuvants. These compositions are also subject matter of the present invention.

The proteins described in this application or proteins identified by the method described above can be used to develop modification-specific diagnostic tools such as antibodies or phages or other substances. The proteins or useful fragments can be used to develop protein chips or other solid-phase screening devices for high throughput screens.

The proteins identified by this technique are potential targets for diseases associated with apoptosis. Such diseases are tumours which can be associated with identified proteins as GAP SH3 binding protein (NM_{—}005754), Baf-57 (4507089), CAF-1 (422892), CBF-beta (2498753), AOP-1 (5802974), SYT interacting protein SIP (5454064), PA1-G (4505587), CRHSP-24 (4583307), FUSE binding protein 1 (4503801), HDGF (4758516), HCA56 (7678701), alpha NAC (NM_{—}005594), ARDH (X77588), DEAD box protein retinoblastoma (NM_{—}004939), HSP-105 (NM_{—}006644), IGF-II mRNA binding protein 1 (NM_{—}006546), IGF-II mRNA binding protein 3 (AF117108), RAD 21 (X98294), RAD 23 homolog B (NM_{—}002874), thioredoxin like protein (NM_{—}004786), hnRNP A/B (4758542), HnRNP A0 (8134660), hnRNP A1 (296650), hnRNP A2/B1 (565643), hnRNP A3 (6164674), hnRNP C1/C2 (4758544), hnRNP D (870743), hnRNP E1 (2134737), hnRNP F (452048), hnRNP H (347314), hnRNP I (4506243), hnRNP K (4504453), hnRNP L (4557645), hnRNP M (5174611), hnRNP R (2697103), Apobec-1 interacting protein (1814274), JKTBP1 (2780748), SRp30c (4506903), p54nrB (1895081), SF2p33 (ASF-2, 179074), SF SC35 (35597), NMP200 (5689738), splicing factor PTB (4826998), splicing factor 1 (1620403), KH-type splicing regulatory protein (FUSE binding protein 2, 2460200), DAZ associated protein (9506537), elongation initiation factor 3 subunit 4 (2460200), NS-associated protein 1 (5453806), nucleolin (4885511), poly(A)-binding protein cytoplasmic 4 (4504715), Ras-GAP SH3 binding protein (3098601), an unnamed protein product (7023323), chondrosarcoma associated protein 2 (5901878), ELAV-like protein 1 (4503551), SKI-interacting protein 1 (2500813), prohibitin (464371), nucleophosmin (114762), T-complex protein 1 beta subunit (1871210), heterochromatin protein p25 (5803076), KIAA1470 (7959201) and cAbl (125135). Further diseases are viral infections like HIV infection which can be associated with identified proteins as Tat binding protein-1 (4506211), CBF-beta (2498753) and EIF-5A (4503477). Further diseases are neurodegenerative diseases like Alzheimer's disease and Parkinson's disease which can be associated with identified proteins as HCD2 (4758504), AOP-1 (5802974), thioredoxin-related protein of 32 kDa (4759274), ERp37 (4885359), cGMP dependent protein f kinase (6225588), VDAC-3 (5032221), HSP105 (5729879) and CRHSP-24 (4583307). Further diseases are ischemic stroke, heart failure and arthritis, which can be associated with identified protein AOP-1 (5802974), VDAC-3 (5032221), HSP105 (5729879), CRHSP-24 (4583307) and PAF acetylhydrolase (4505587).

Therefore the lack of expression or over-expression can be indicative of a disease and thus has diagnostic implications. The genes of the identified proteins can be used to develop DNA-chips or other DNA-or RNA-based screening devices (PCR, RT-PCR) to screen cells or tissues for the differences in the mRNA levels of the identified genes.

We could show that caspases cleave GAP SH3 binding protein after amino acids D168 (amino acid sequence EVVPDDSGT, cleavage site underlined) and D422 (amino acid sequence AREGDRRDN). Cleavage at D168 separates the N-terminal fragment containing the nuclear transport factor 2 motif (NTF2-motif) from the protein. Cleavage at D422 separates the two RNP-motifs (RNP1 amino acids 341 to 346, RNP2 amino acids 378 to 385) from the RGG-motif (amino acids 429 to 461). We could further show that cleavage at D422 is sensitive to RNA binding suggesting that the RNAse activity of GAP SH3 binding protein is modulated by caspase cleavage. We further identified the ubiquitin C-terminal hydrolase related polypeptide (NM_{—}009462) and the GAP SH3 binding protein itself as binding partners for the N-terminal caspase cleavage product comprising amino acids 1 to 168 of GAP SH3 binding protein.

Thus, GAP SH3 binding protein or fragments thereof generated during apoptosis can be used to generate diagnostic tools such as cleavage specific antibodies or phages or other tools useful for large scale screening. The gene of the GAP SH3 binding protein can be used to develop DNA-chips or other DNA- or RNA-based screening devices (PCR, RT-PCR) to screen cells or tissues for the differences in the mRNA levels of the identified genes or to screen for mutations in the caspase cleavage site of the GAP SH3 binding protein.

GAP SH3 protein or fragments generated during apoptosis can be used to screen drugs which activate or inhibit their activity. This activity may be modification of the activity of Ras-GAP which modifies the activity of the Ras-oncoprotein or other GTPases. The activity may be the RNA-binding or RNAse activity elicted by the apoptosis-specific modification of GAP SH3 binding protein. This activity may be any activity elicited by the modification of the protein during apoptosis. For example, this activity may be the binding to ubiquitin C-terminal hydrolase related polypeptide (UCHRP) or related proteins. A consequence of binding to UCHRP or related proteins may be the modification of cell differentiation in tumour genesis. GAP SH3 binding protein and binding partners might play an important role in tumour formation and metastasis formation. Alternatively, this activity may be the binding of GAP SH3 binding protein (dimerisation, multimerisation) which might be a prerequisite for a possible function of GAP SH3 binding protein in tumourgenesis and/or metastasis formation.

GAP SH3 binding protein is therefore potentially involved in the growth control of cells. Tumours can over-express or lack GAP SH3 binding protein or produce a modified GAP SH3 binding protein. Tumours can be defective in the RNA-modifying activity of GAP SH3 binding protein. Tumours can be defective of or constitutively bind interacting proteins like UCHRP or related proteins or GAP SH3 binding protein. Signals transduced via UCHRP or related proteins or GAP SH3 binding protein dimers or multimers or any interaction protein might trigger tumour genesis or metastasis formation. Drugs which interfere with constitutive GAP SH3 binding protein activity or which activate GAP SH3 binding protein activity or which interfere with binding or interacting proteins are useful for therapy of such diseases.

Alzheimer's disease is associated with premature apoptosis of neuronal cells. Neuronal cells of Alzheimer patients are characterised by the accumulation of β-amyloid precursor protein which is known to interact with HCD2 (Yan et al., 19937, Nature, 389, 689-695). HCD2 was found to translocate from the cytosol to the nucleus (compare Tables 4 and 5) and is thereby modified, probably by phosphorylation. HCD2 translocation can be the cause of β-amyloid precursor accumulation and thus a promoter of Alzheimer's disease.

HCD2 or the modified HCD2 generated during apoptosis can be used to generate diagnostic tools such as modification-specific antibodies or phages or other tools useful for large scale screening. The gene of the HCD2 protein can be used to develop DNA-chips or other DNA- or RNA-based screeing devices (PCR, RT-PCR) to screen cells or tissues for the differences in the mRNA levels of the identified genes or to screen for mutations in the modification site (phosphorylation site) of the HCD2 protein.

HCD2 or the modified HCD2 generated during apoptosis can be used to screen drugs which activate or inhibit their activity and which are useful in prevention and/or treatment of Alzheimer's disease. This activity can be binding and/or sequestration of the β-amyloid precursor protein and prevention of apoptosis in neuronal cells or other cells. The activity can be the enzymatic activity of the HCD2 which is preferably any activity associated with prevention of apoptosis and more preferably a dehydrogenase activity (34). This activity can be any activity elicited by the modification (e.g. translocation) of the protein during apoptosis.

AOP-1 protects radical-sensitive proteins (enzymes) from oxidative damage. Oxidative stress has been demonstrated to induce apoptosis in different cell types. In addition, oxidative stress is involved in several diseases. AOP-1 as protecting molecule can be used to prevent-and/or to treat diseases related to oxidative stress like ischemic stroke, arthritis, heart failure, Parkinson's disease, Alzheimer's and amyotrophic lateral sclerosis (ALS). The cleavage and/or translocation of AOP-1 (see Table 5) from the mitochondria to the nucleus is accompanied with a change in its activity. AOP-1 or the modified AOP-1 generated during apoptosis can be used to generate diagnostic tools such as modification-specific antibodies or phages or other tools useful for large scale screening. The gene of the AOP-1 protein might be used to develop DNA-chips or other DNA- or RNA-based screening devices (PCR, RT-PCR) to screen cells or tissues for the differences in the mRNA levels of the identified genes or to screen for mutations in the modification site (cleavage site) of the AOP-1 protein.

AOP-1 or the modified AOP-1 generated during apoptosis can be used to screen drugs which modify their activity. This activity can be protection from radical induced damage of proteins and therapy of the diseases outlined above. The activity can be the enzymatic activity of the AOP-1 which is preferably any activity associated with prevention of apoptosis and more preferably a peroxide reductase activity. This activity can be any activity elicited by the modification or/and translocation of the protein during apoptosis. The gene of the AOP-1 protein can be used for gene therapy of diseases associated with radical induced protein damage followed by apoptosis.

The c-Abl tyrosine kinase has been shown to posses oncogenic activity. It is activated in response to genotoxic and oxidative stress. Cells deficient in c-Abl or expressing dominant negative forms of c-Abl exhibit an attenuated apoptotic response to different genotoxic agents.

We could show that cells treated with apoptosis inducing agents like TNFα, Fas, Etoposide or cis-platin cleave nuclear and cytosolic c-Abl. Caspases were identified by inhibitor studies and in vitro cleavage assays as the proteases responsible for the cleavage of cAbl. These caspases include caspase-3, caspase-8, and caspase-10. We could demonstrate that cleavage by caspase activates cAbl kinase. Amino acids D546 (amino acid sequence PELPTKTRTSRRAAEHRDTTDVPEMPHSKGQGESD, cleavage site underlined), D655 (PLDTADPAKSP) and D939 (ATSLVDAVNSD) were identified as cleavage sites of the human 1A form of c-Abl (p00519). A cleavage site corresponding to the D546 is also present in murine homolog cAbl Type I (sequence PELPTKTRTCRRAAEQKDAPDTPELLHTKGLGESD, J02995.1, P00520), whereas in the Abl related kinase (Arg, p42684) none of the cleavage sites is conserved. Furthermore, in the transforming viral homolog vAbl (P00521) there exists a sequence (AA786-789) homolog to the D939 cleavage site in human cAbl 1A (p00519). In contrast to this, the other two cleavage site (D546 and D655) are not conserved, even more the homology between the murine cAbl and the viral vAbl is disrupted exactly at the caspase cleavage site D546 (vAbl sequence PELPTKTRTCRRAAEQKASPPSLTPKLLRRQVTASPS). Thus, vAbl may circumvent apoptotic death of infected cells by its inability to be processed by caspases. The cleavage of cAbl leads to the release of a Src homolog N-terminal and two C-terminal fragments.

Caspase cleavage of cAbl at D939 leads to the release of cAbl from the cytoskeleton. Subsequent cleavage at D546 and D655 activates cAbl kinase function. By overexpression of a mutant, deficient in D546 and D655 cleavage sites, we could inhibit TNFalpha induced apoptosis in Hela cells. The release of cAbl from the cytoskeleton by caspase cleavage at D939 is essential for this phenotype, as a total cleavage mutant (D546N, D655N, D939N) failed to inhibit apoptosis. Thus, the lack of caspase cleavage site D546 and D655 in cAbl renders cells apoptosis resistant suggesting that cleavage of cAbl is an essential process in apoptosis signalling. By inhibiting caspase cleavage at D546 and D655, diseases with aberrant apoptosis (for example neurodegenerative diseases) can be treated.

The fusion between Bcr and Abl (Bcr/Abl) has been implicated in chronic myelogenic leukaemia. 95% of the patients carry the fusion. The Bcr/Abl fusion localises to the cytosol and exerts a constitutive kinase activity. The caspase cleavage sites identified for cAbl are conserved in Bcr/Abl. Thus, caspase cleavage or lack of cleavage of Bcr/Abl might be an important event in chronic myelogenic leukaemia.

The gene of cAbl or Bcr/Abl can be used to develop DNA-Chips or other DNA- or RNA-based screening devises (PCR, RT-PCR) to screen cells or tissues for the differences in the mRNA levels of the identified genes or to screen for mutations in the caspase cleavage sites of cAbl or Bcr/Abl. cAbl or Bcr/Abl proteins or fragments generated during apoptosis might be used to screen drugs which activate or inhibit their activity. This activity can be a kinase activity, interaction with other proteins, lack of interaction with proteins leading to oncogenic transformation or induction of apoptosis. This activity can be any activity elicited by the modification of the proteins during apoptosis.

cAbl or Bcr/Abl or domains of these proteins might be used to generate specific therapeutic approaches which lead to the cleavage of these proteins and the induction of apoptosis.

cAbl and Bcr/Abl are involved in the growth control of cells. Tumours might over-express or lack cAbl or produce a modified cAbl. The modification can involve the caspase cleavage sites. Tumours might be defective in processing of cAbl or Bcr/Abl. Drugs which interfere with constitutive cAbl or Bcr/Abl activity or which activate cAbl or Bcr/Abl are useful for therapy of diseases, particularly tumours. The cAbl protein or fragments generated during apoptosis can be used to generate antisera, monoclonal antibodies or phages specific for the detection of modified cAbl or Bcr/Abl. Antisera, monoclonal antibodies or phages specific for the detection of modified cAbl or Bcr/Abl can be used for diagnosis of diseases, particularly of tumours.

Caspase cleavage of substrates like cAbl induces the activation of apoptosis. Lack of caspase cleavage in key substrates of apoptosis as shown in the cAbl cleavage-resistant mutant leads to apoptosis resistance. The specific cleavage of a key substrate might be used as therapeutic approach to either induce or inhibit apoptosis in diseases such as proliferative diseases or degenerative diseases. Possible approaches include specific drugs or peptides or antibodies or phages or any substance which block the cleavage of a substrate by caspases. Further approaches include drugs or peptides or antibodies or phages or specific interaction domains of proteins which in connection with proteases (e.g. caspases) are useful to specifically cleave substrates.

p54nrb (1895081) is a nuclear RNA-binding protein with high homology to splicing factors. We found that p54nrb is cleaved by caspases after amino acids D231 (EPMDQLDDEEGLP), D286 (EMEKQQQDQVDRNIK), D422 (APPGPATMMPDGTLGLTP) and after an additional, unidentified site in vitro, and after D422 in vivo. We demonstrated that cleavage after D231, D286 and the unidentified site, but not after D422 is sensitive to RNA-binding suggesting that caspases significantly influence the RNA-binding and -modification function of p54nrb. Alternative splicing of key molecules like caspases, receptors and Bcl-2 family members plays an important role in apoptosis regulation. Thus p54nrb might influence apoptosis by modifying mRNA of regulators of apoptosis. Cleavage of p54nrb might activate or inactivate its RNA-modification activity leading either to inhibition or activation of apoptosis. Alternatively, p54nrb or an activity elicited by p54nrb might be involved in proliferation which is counteracted during apoptosis by caspase cleavage.

p54nrb or RNA binding proteins which act by a similar mechanism as p54nrb might be targets for general apoptosis regulation by RNA modification. Furthermore, p54nrb or RNA binding proteins which act by a similar mechanism as p54nrb might be suitable targets for the therapeutic intervention of proliferative diseases. Purified proteins of these factors or fragments thereof might be used to screen for drugs which inhibit or increase its activity. These factors or fragments generated during apoptosis can be used to generate diagnostic tools such as cleavage specific antibodies or phages or other tools for large scale screening. The genes of these factors might be used to develop DNA-chips or other DNA- or RNA-based screening devices (PCT, RT-PCR, filters) to screen cells or tissues for differences in the mRNA levels of the identified genes or to screen for mutations in their caspase cleavage sites.

We found BAF57 (4507089), CAF-1 p48 (422892), p54nrb (1895081), hnRNP R (2697103), nucleolin (4885511), SF ASF-2 (105294), TF BTF3a (29597), CNF B1 (7020309) to be cleaved by caspases in vitro and in vivo, hnRNP A2/B1 (4758542) and KIAA1470 (7959201) to be cleaved in apopototic cells in vivo. These factors show DNA- or RNA binding activity or are involved in chromatin remodelling and are thus potentially involved in growth control. Cleavage by caspases or other apoptosis related proteases might inactivate these factors to inhibit growth signals during the apoptotic process. Tumours can over-express or lack these factors or express modified forms of these factors. These factors might be suitable targets for therapeutic intervention of proliferative diseases. Purified proteins of these factors or fragments thereof might be used to screen for drugs which inhibit or increase their activity. These factors or fragments generated during apoptosis can be used to generate diagnostic tools such as cleavage specific antibodies or phages or other tools for large scale screening. The genes of these factors might be used to develop DNA-chips or other DNA- or RNA-based screening devises (PCR, RT-PCR, filters) to screen cells or tissues for differences in the mRNA levels of the identified genes or to screen for mutations in their caspase cleavage sites.

We found cGMP-dependent protein kinase (6225588) to be cleaved in apopototic cells in vivo. cGMP-dependent protein kinase (cGDPK) is involved in NO signalling which is an important signalling pathway in ischemic stroke, heart failure, neuro-degenerative diseases like Parkinson's disease and Alzheimer's disease. cGDPK is particularly important in NO-mediated smooth muscle cell regulation and is implicated in NO-mediated vasodilatation. Therefore cGDPK might be involved in arteriosclerosis and other vascular diseases. Modulation of cGDPK activity during apoptosis might be an important signal for the development of these diseases.

cGDPK might be a suitable target for therapeutic intervention of ischemic stroke, heart failure, neuro-degenerative diseases like Parkinson's disease and Alzheimer's disease, arteriosclerosis and other vascular diseases. Purified cGDPK or fragments might be used to screen for drugs which inhibit or increases its activity. Purified cGDPK or fragments might be used to screen specific drugs or peptides or antibodies or phages or any substance which block the cleavage of cGDPK by caspases. Further approaches include drugs or peptides or antibodies or phages or specific interaction domains of proteins which in connection with proteases like caspases are useful to specifically cleave cGDPK. cGDPK or fragments generated during apoptosis can be used to generate diagnostic tools such as cleavage specific antibodies or phages or other tools for large scale screening. The gene of cGDPK might be used to develop DNA-chips or other DNA- or RNA-based screening devises (PCR, RT-PCR, filters) to screen cells or tissues for differences in the mRNA levels of the identified genes or to screen for mutations in their caspase cleavage sites.

SYT-interacting protein SIP (5454064), IGF-II mRNA binding protein 1 (5729882), IGF-II mRNA binding protein 3 (4191612), HCA56 (7678701), chondrosarcoma-associated protein 2 (5901878), ELAV-like 1 (4503551), SKI-interacting protein (6912675), heterochromatin protein p25 (5803076) and Rad 23 (4506387) were found only in patterns of normal but not of apoptotic cells. These proteins are therefore possibly processed during apoptosis by caspases or other proteases. These factors display DNA- or RNA binding activity or are involved in chromatin remodelling or interact with potential oncogenes or are involved in DNA-repair or are known to be expressed in tumours and are thus potentially involved in growth control. Cleavage by caspases or other apoptosis related proteases might inactivate these factors to inhibit growth signals and DNA repair during the apoptotic process. Tumours can over-express or lack these factors or express modified forms of these factors. These factors might be suitable targets for therapeutic intervention of proliferative diseases. Purified proteins of these factors or fragments thereof might be used to screen for drugs which inhibit or activate their activity. These factors or fragments generated during apoptosis can be used to generate diagnostic tools such as cleavage specific antibodies or phages or other tools for large scale screening. The genes of these factors might be used to develop DNA-chips or other DNA- or RNA-based screening devises (PCR, RT-PCR, Filters) to screen cells or tissues for differences in the mRNA levels of the identified genes or to screen for mutations in their caspase cleavage sites.

FUSE-binding protein 1 (4503801) and 2 (4504865), DEAD-box protein retinoblastoma (4826686), CBF beta/PEBP2 (2498753), nucleophosmin (114762), T-complex protein 1 beta subunit (TCP-1, 1871210), hepatoma derived growth factor (HDGF, 4758516) and RAD21 (1620398) are factors which potentially translocate during apoptosis. Translocation is an important mechanism of apoptotic signalling. These factors display DNA- or RNA-binding activity or are known to be expressed in tumours and are thus potentially involved in growth control. RAD21 is involved in DNA repair which is of particular importance in fast growing cells. Translocation of RAD21 might prevent DNA repair in apoptotic cells. Tumours can over-express or lack these factors or express modified forms of these factors. These factors might be suitable targets for therapeutic intervention of proliferative diseases. Purified proteins of these factors or fragments thereof might be used to screen for drugs which inhibit or activate their activity. These factors or fragments generated during apoptosis can be used to generate diagnostic tools such as cleavage specific antibodies or phages or other tools for large scale screening. The genes of these factors might be used to develop DNA-chips or other DNA- or RNA-based screening devises (PCR, RT-PCR, Filters) to screen cells or tissues for differences in the mRNA levels of the identified genes or to screen for mutations in their caspase cleavage sites.

In the course of the works leading to the present invention several caspase cleavage sites have been discovered. Such cleavage sites are summarized in Table 9. The cleavage sites generally include four amino acids, the last amino acid being D.

The present invention, therefore, also relates further to uses and methods related to such caspase cleavage site.

In a first aspect, the knowledge about the cleavage sites can be used to generate recombinant proteins with modified cleavage sites. Such proteins cannot be cleaved by caspases anymore and can be used for example for screening or development of pharmaceuticals.

In a second aspect the knowledge about the cleavage sites can be used within the design and/or screening for substances that inhibit or modulate caspase cleavage of proteins that contain caspase cleavage site. The screening can be done for example in a first step by a data base search and in a second step by performing assays wherein the candidate inhibitor or modulator compounds are evaluated using a peptide or protein containing such cleavage site. In a preferred embodiment, the cleavage site is contained in or associated with a reporter gene. In such combination of cleavage site and reporter gene the cleavage can be easily surveyed. Useful reporter genes are known to the man in the art.

In a third aspect a peptide or a protein containing a caspase cleavage site can be used as a diagnostic tool to screen for caspase activity, e.g. in cells or cell extracts, and/or to determine the effectivity of caspase cleavage inhibiting and/or modulating substances.

Recombinant proteins or peptides containing such cleavage sites are also encompassed by the present invention.

### The present invention is to be further illustrated by the following figures and examples.

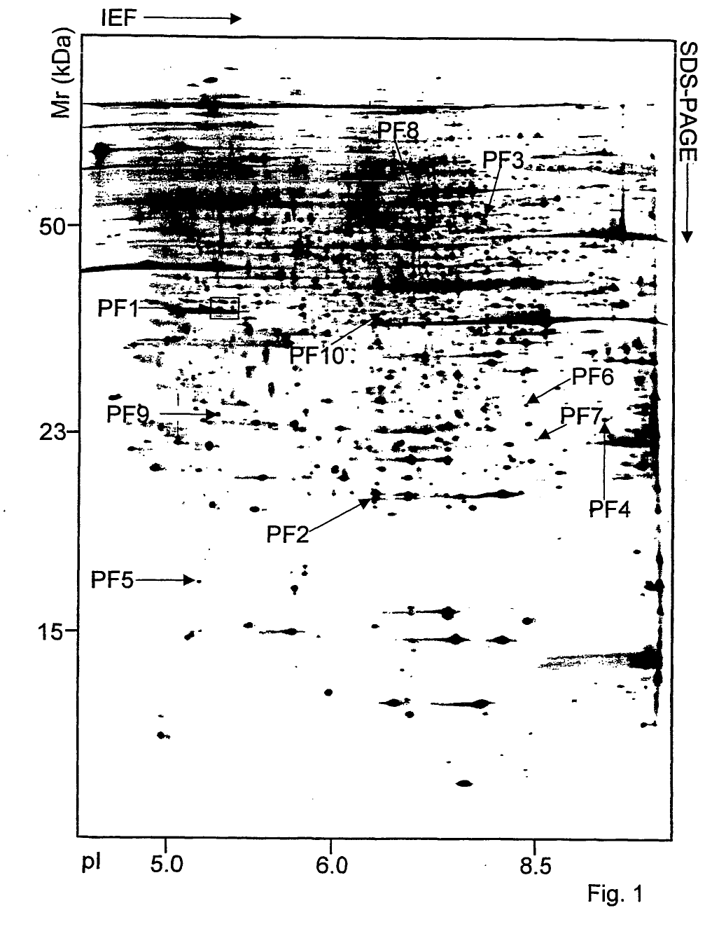

FIG. 1

2-DE gel of Fas-induced Jurkat T-cells (see Table 3).

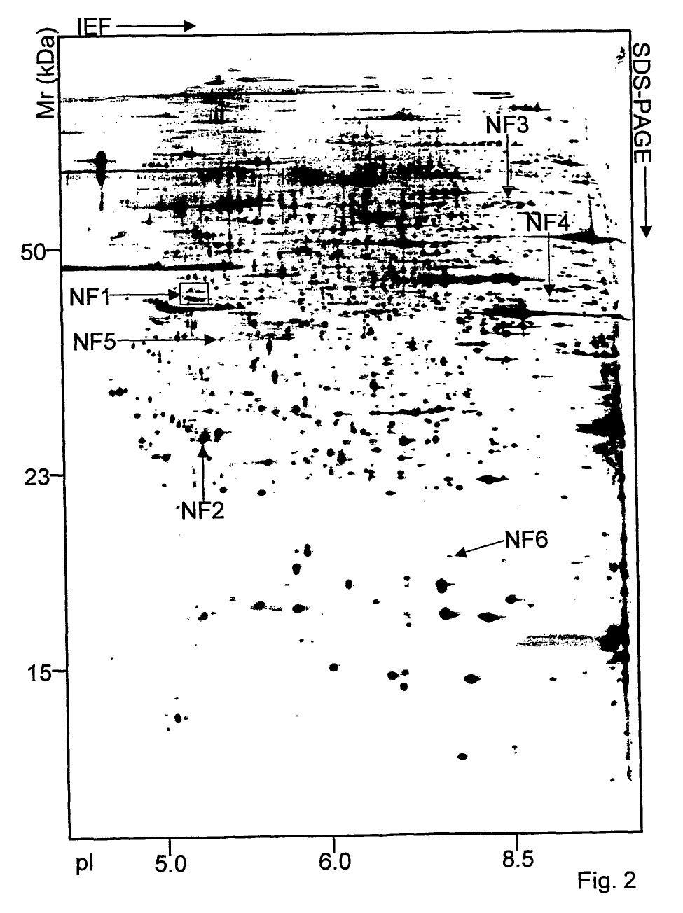

FIG. 2

2-DE gel of Jurkat T-cells (control, see Table 3).

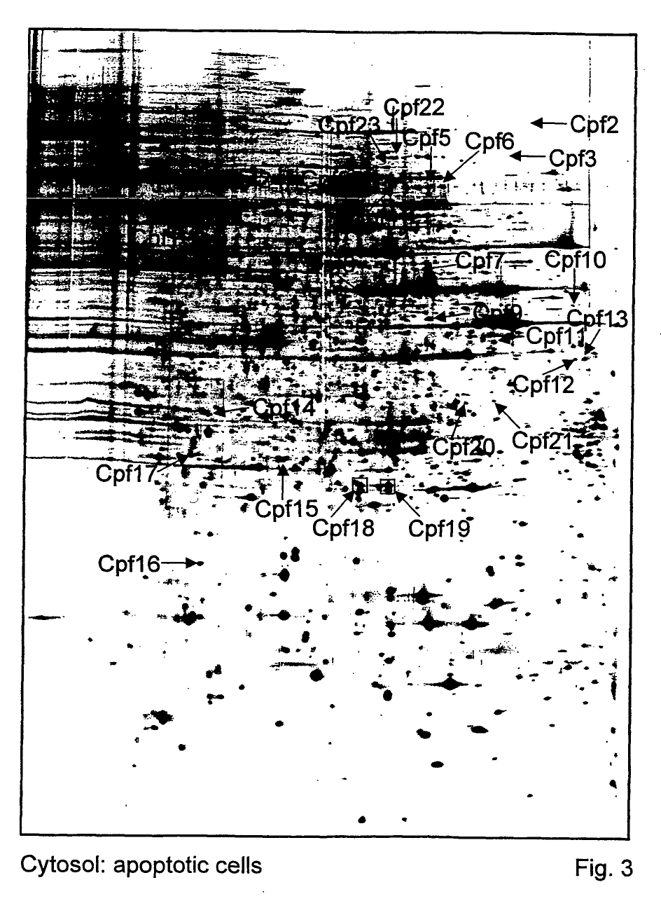

FIG. 3

2-DE gel of the cytosolic compartment of Fas-induced Jurkat T-cells (see Table 4).

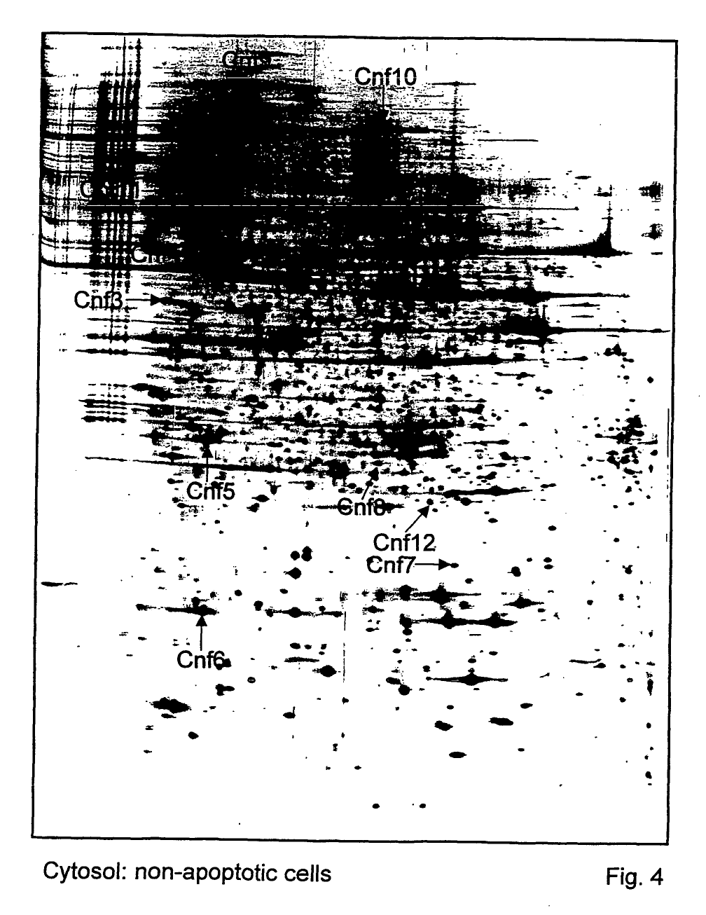

FIG. 4

2-DE gel of the cytosolic compartment of Jurkat T-cells (control, see Table 4).

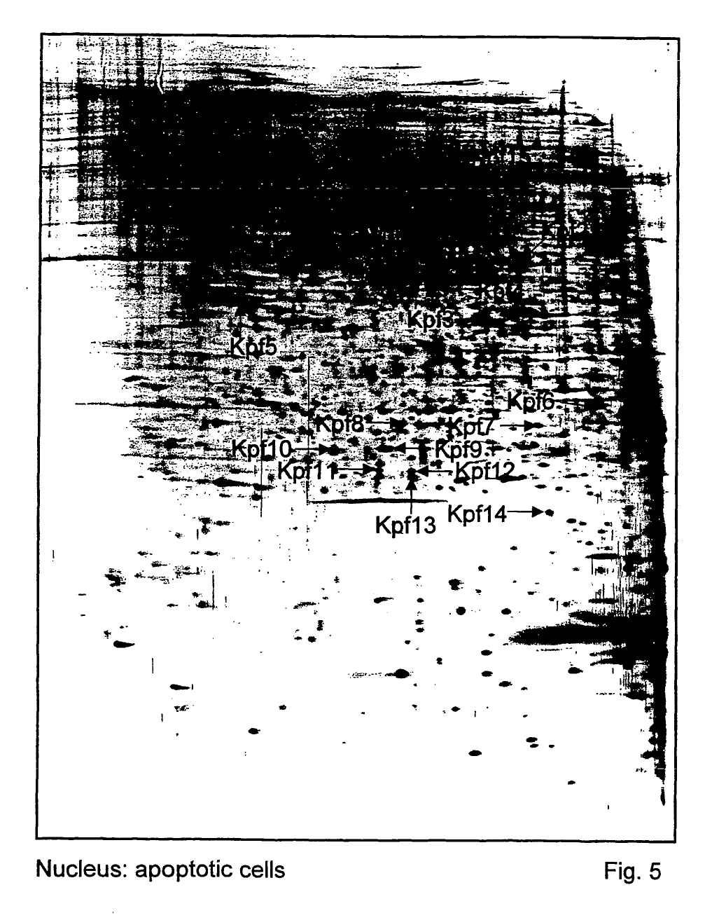

FIG. 5

2-DE gel of the nucleic compartment of Fas-induced Jurkat T-cells (see Table 5).

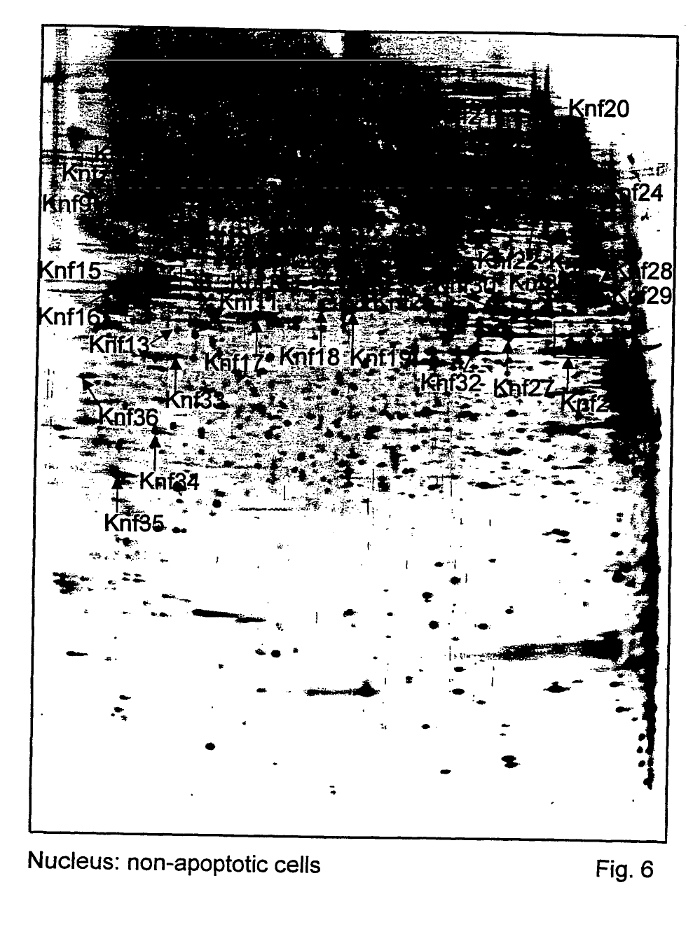

FIG. 6

2-DE gel of the nucleic compartment of Jurkat T-cells (control, see Table 5).

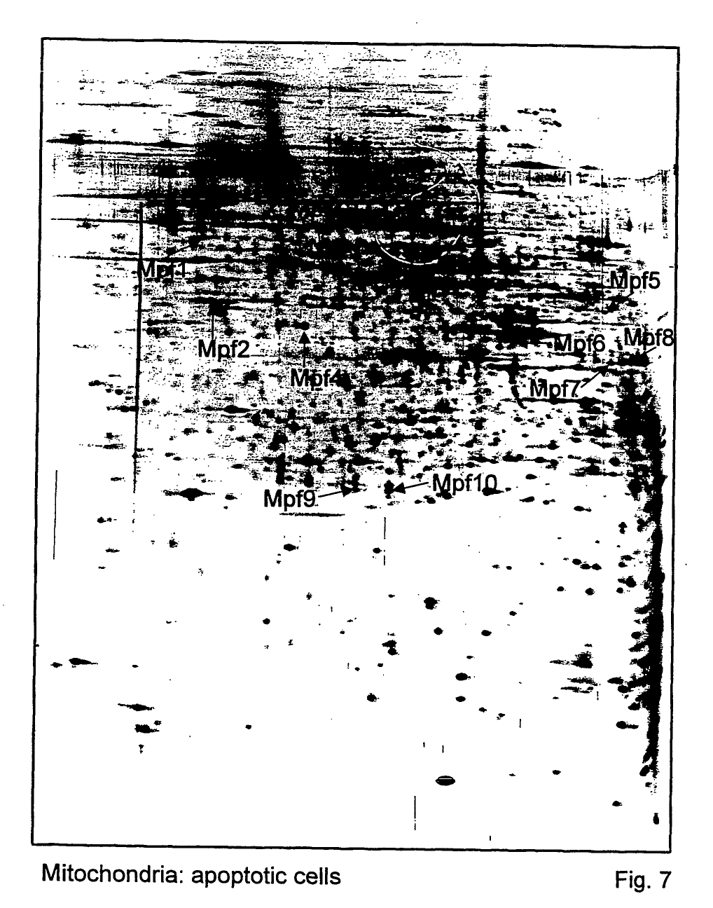

FIG. 7

2-DE gel of the mitochondrial compartment of Fas-induced Jurkat T-cells (see Table 6)

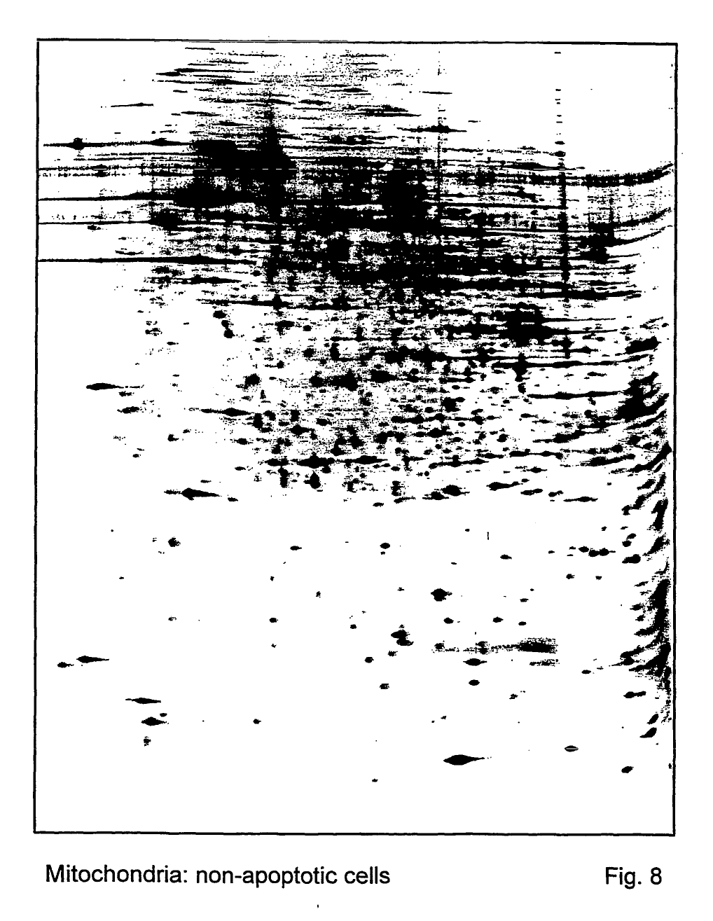

FIG. 8

2-DE gel of the mitochondrial compartment of Jurkat T-cells (control, see Table 6)

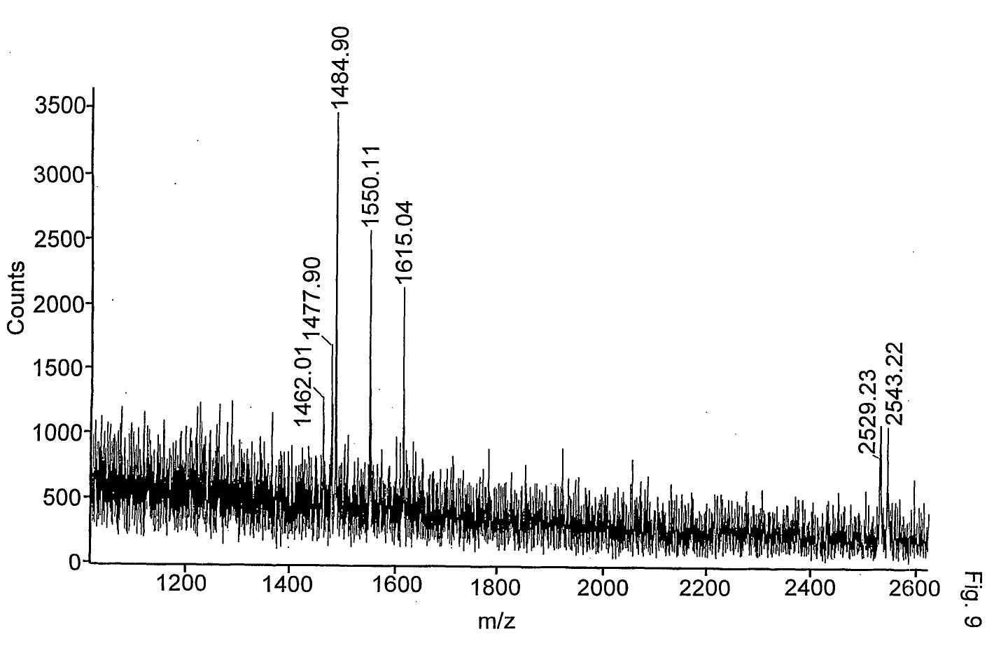

FIG. 9

Peptide mass fingerprinting of unknown protein called PFC6D (see Table 3). The peptide is characterized by fragments with the following masses: 1462.01, 1477.9, 1484.9, 1550.11, 1615.04, 2529.33, 2543.22 dalton.

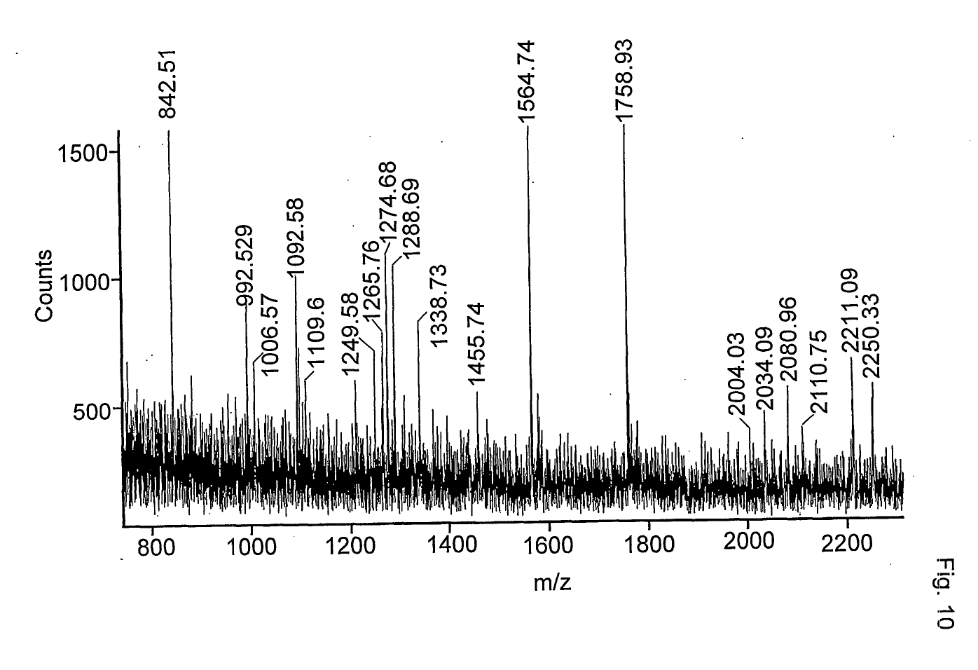

FIG. 10

Peptide mass fingerprinting of unknown protein called KPF1 (see Table 5). The peptide is characterized by fragments with the following masses: 842.15, 992.529, 1006.57, 1092.58, 1109.6, 1274.68, 1288.68, 1265.76, 1249.58, 1338.73, 1455.74, 1564.74, 1758.93, 2004.03, 2034.09, 2080.96, 2110.75, 2211.09 and 2250.33 dalton.

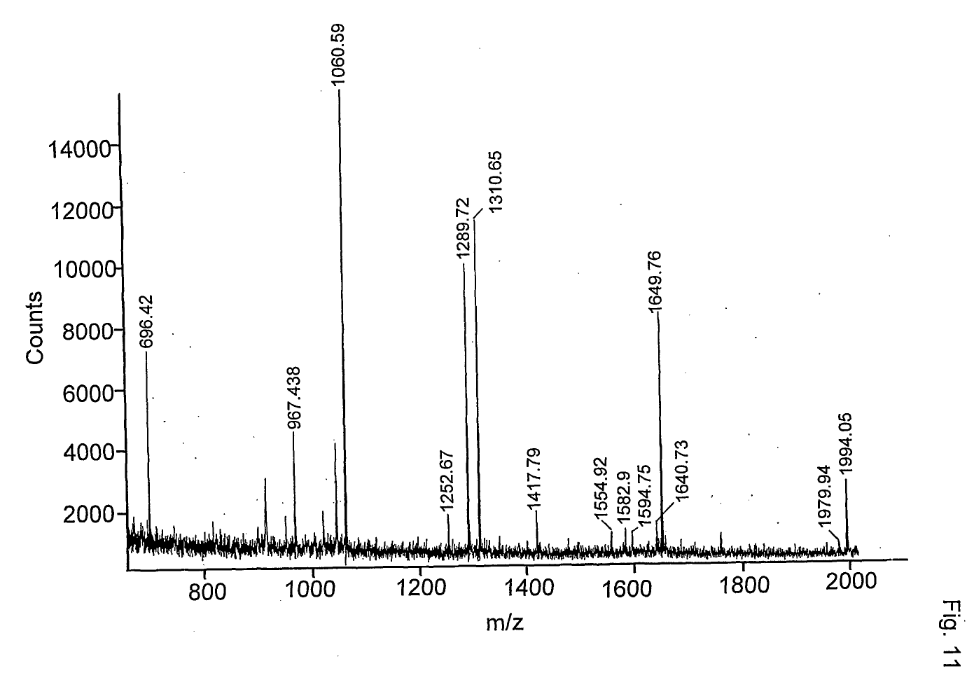

FIG. 11

Peptide mass fingerprinting of unknown protein called KNFE3 (see Table 5). The peptide is characterized by fragments with the following masses: 696.42, 967.438, 1060.59, 1252.67, 1289.72, 1310.65, 1417.79, 1554.92, 1582.9, 1594.75, 1640.73, 1649.76, 1979.94, 1994.05 dalton.

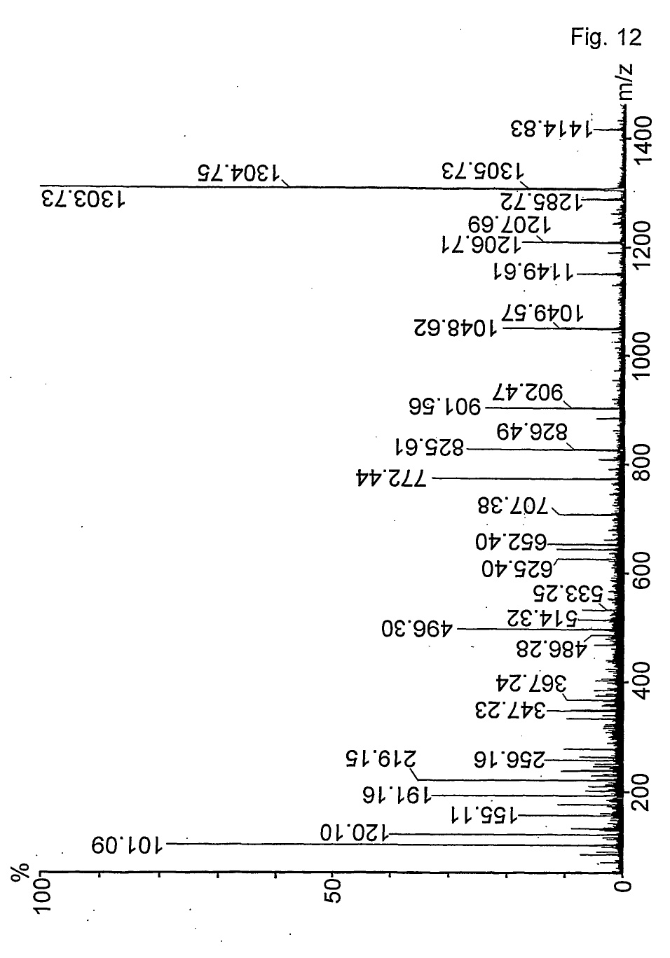

FIG. 12

The partial sequence TPGT(F/Mox)E of the protein KNFE3 was obtained by ESI-MS/MS of the 1649,79 dalton fragment.

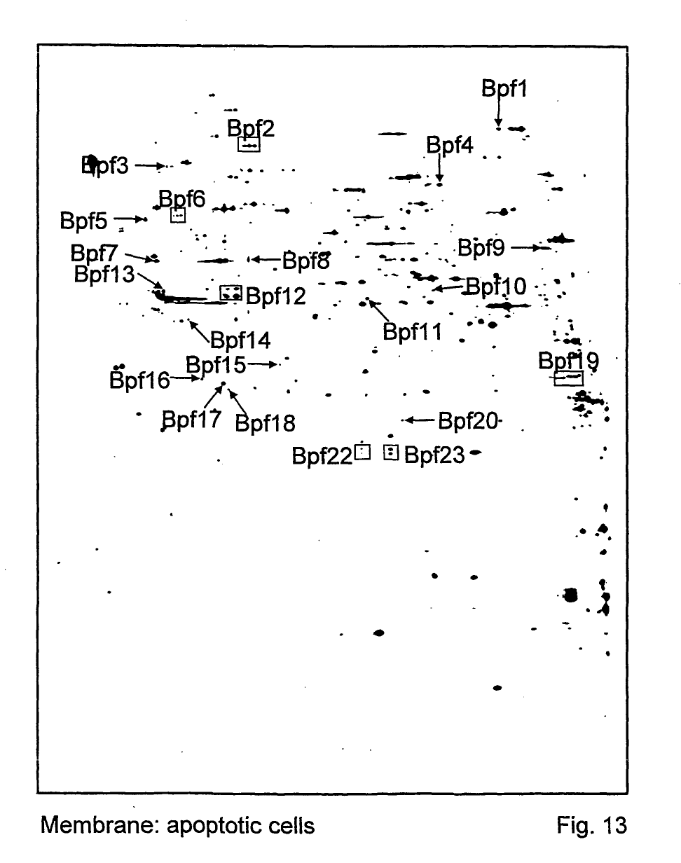

FIG. 13

2-DE gel of the membrane compartment of Fas-induced Jurkat T-cells (cf. Table 7)

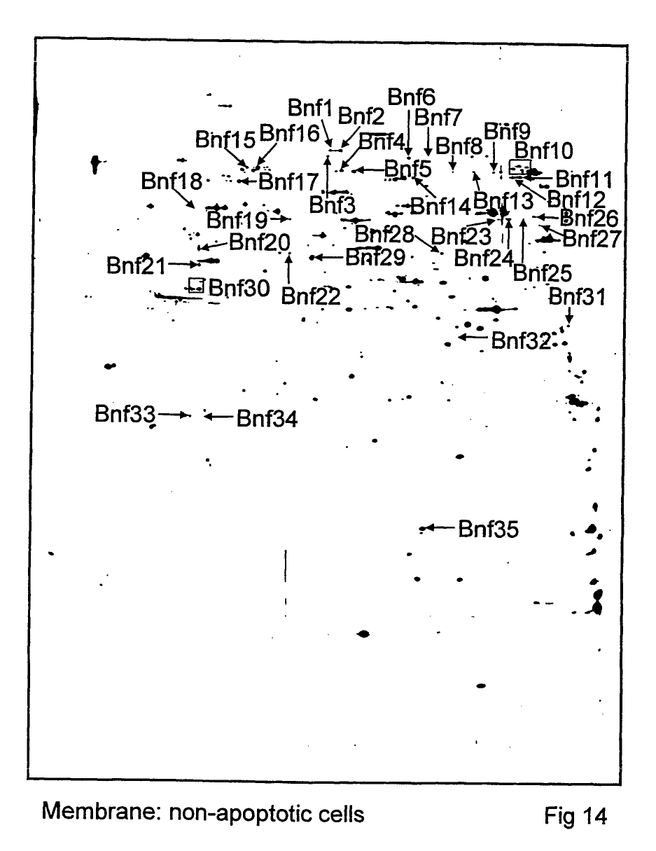

FIG. 14

2-DE gel of the membrane compartment of Jurkat T-cells (control, cf. Table 7)

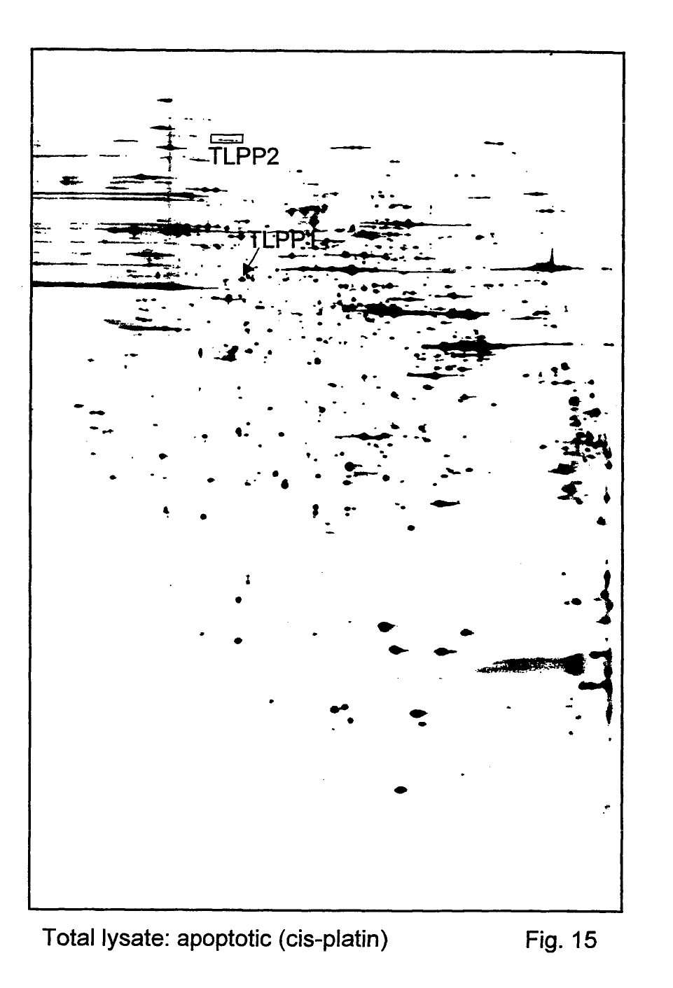

FIG. 15

2-DE gel of the total cell lysate of cis-platin induced apoptotic Jurkat T-cells (cf. Table 8).

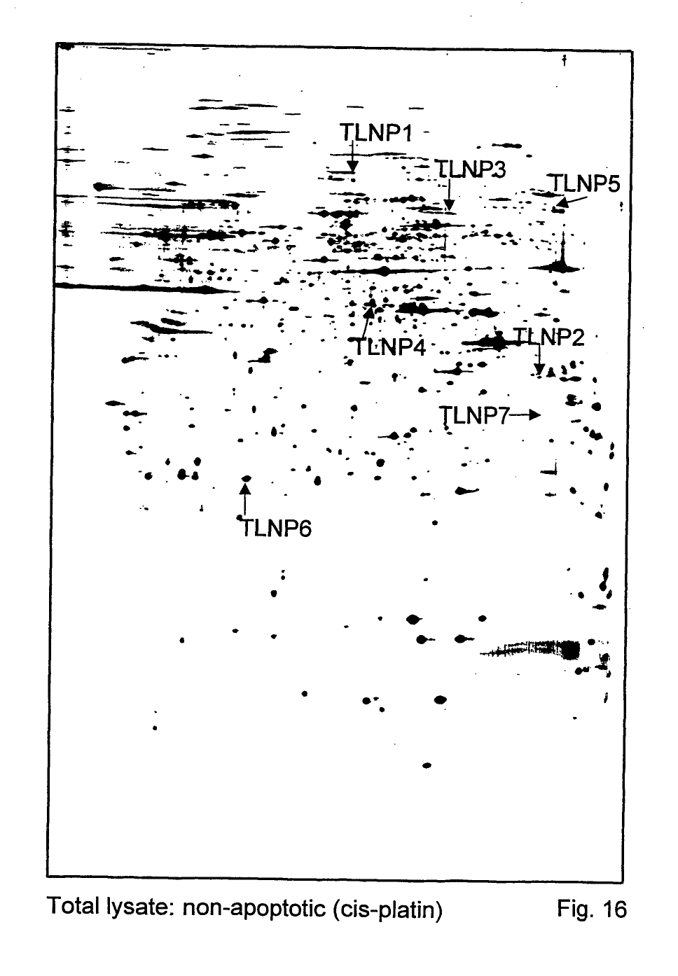

FIG. 16

2-DE gel of the total cell lysate of Jurkat T-cells (control, cf. Table 8).

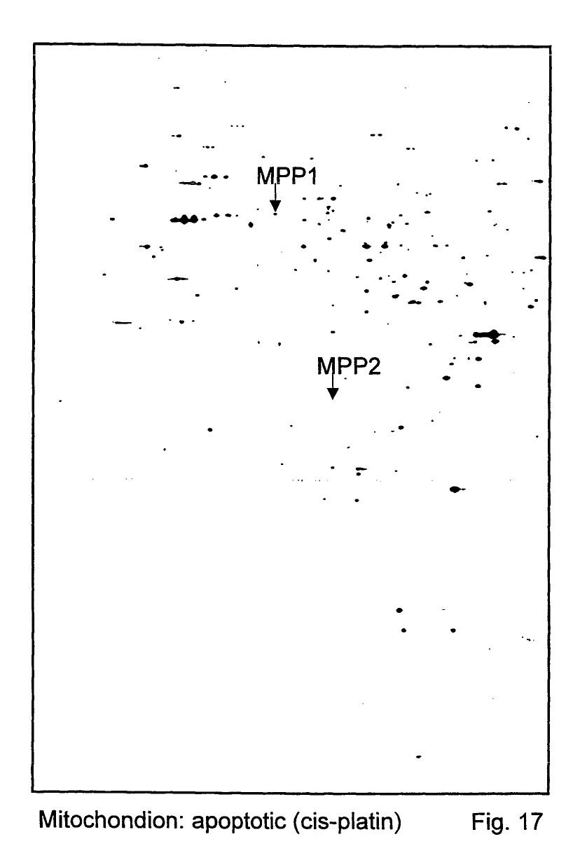

FIG. 17

2-DE gel of the mitochondrial compartment of cis-platin induced Jurkat T-cells (cf. Table 8). The control (mitochondrial compartment of non-induced T-cells) is not shown.

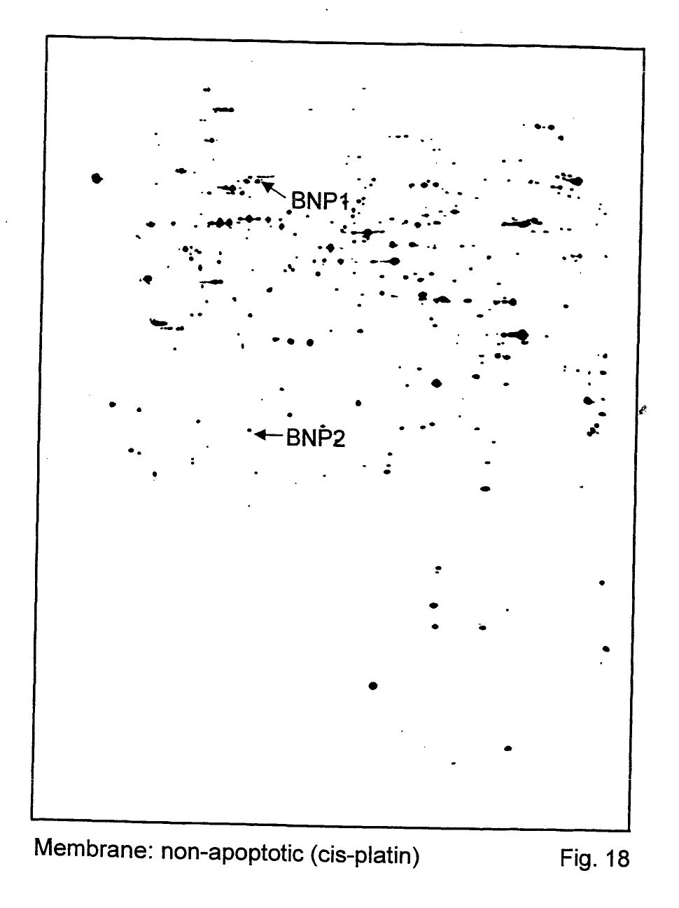

FIG. 18

2-DE gel of the membrane compartment of Jurkat T-cells (cf. Table 8). The membrane compartment of cis-platin induced Jurkat T-cells is not shown.

### EXAMPLE

1. Materials and Methods

1.1 Cell Culture

The Jurkat T-cell line E6 (ATCC TIB 152) was maintained in RPMI tissue culture medium (Gibco BRL, Karlsruhe, Germany) supplemented with 10% fetal calf serum (Gibco BRL, Karlsruhe, Germany) and penicillin (100 U/ml)/streptomycin (100 μg/ml) (Gibco BRL, Karlsruhe, Germany) at 37° C. in 5.0% CO₂.

1.2 Induction of Apoptosis

Apoptosis was induced to 2×10⁶ Jurkat T-cells for 6 h at 37° C. in 5.0% CO₂ by 250 ng/ml αCD95 (clone CH11) (Immunotech, Marseille, France) or for 16 h at 37° C. in 5.0% CO₂ by 60 μM cis-platinum(II)diaminedichloride (cis-platin, Sigma, Deisenhofen, Germany) in DMSO. 1 μg/ml cycloheximide was added to the control- and Fas induced cells, 0.5 μg/ml cycloheximide was added to the control- and cis-platin induced cells.

1.3 Separation of the Compartments

Approximately 1×10⁸ Jurkat T cells were centrifuged for 10 min at 1300 U/min at room temperature in a Megafuge 1.0R (Heraeus, Hanau, Germany). The supernatant was discarded and the pellet was washed twice with 10 ml PBS (GibcoBRL, Karlsruhe, Germany) and once with MB buffer (400 mM sucrose, 50 mM Tris, 1 mM EGTA, 5 mM 2-mercaptoethanol, 10 mM potassium hydrogenphosphate pH 7.6 and 0.2% BSA) and centrifuged as above. The pellet was suspended in MB buffer (4 ml/10⁸ cells) and incubated on ice for 20 min. Subsequently the cells were homogenized and centrifuged at 3500 U/min for 1 min at 4° C. (Rotor SS-34; Sorvall RC5B, Hanau, Germany). The supernatant contained the mitochondria/cytosol/membranes and the pellet enclosed the nucleus.

The mitochondrial fraction was pelleted by centrifugation at 8600 U/min for 10 min at 4° C. (Rotor SS-34; Sorvall RC5B, Hanau, Germany). The supernatant contained the cytosol and membranes.

The pellet was suspended in MSM buffer (10 mM potassium hydrogenphosphate pH 7.2, 0.3 mM mannitol and 0.1% BSA) (0.4 ml/10⁸ cells) and purified by sucrose gradient centrifugation in 10 ml SA buffer (1.6 M sucrose, 10 mM potassium hydrogenphosphate pH 7.5 and 0.1% BSA) at 20000 U/min, 1 hour, 4° C. (Rotor SW-28; Beckman L8-70M Ultracentrifuge, München, Germany). The interphase which contained the mitochondria was collected, suspended in 4 volumes of MSM buffer and centrifuged again at 15500 U/min for 10 min. at 4° C. (Rotor SS-34; Sorvall RC5B, Hanau, Germany). The pellet was suspended in MSM buffer without BSA and could be stored at −70° C.

The supernatant with the cytosol and membrane was centrifuged at 100000 U/min, 20 min, 4° C. (Rotor TLA120.2 rotor, Ultracentrifuge Optima TLX, Beckman, München, Germany). The pellet contained the membranes.

The pellet with the nucleus was suspended in 5 ml PBS and centrifuged for 2 min at 3500 U/min at 4° C. (Rotor SS-34; Sorvall RC5B, Hanau, Germany). The pellet was suspended in NB buffer (10 mM Hepes pH 7.4, 10 mM KCl, 2 mM dithiothreitol (DTT) and 1 mM Pefabloc) (1 ml/10⁸ cells) and incubated for 1 hour on ice, subsequently homogenized and applied to 10 ml 30% sucrose in NB buffer. After the centrifugation with the Megafuge 1.0R (Heraeus, Hanau, Germany) at 2000 U/min for 10 min at 4° C., the pellet was washed twice with 6 ml NB buffer, centrifuged as above, suspended in 1 ml NB buffer, and centrifuged again at 10000 U/min for 10 minutes at 4° C. (Rotor SS-34; Sorvall RC5B, Hanau, Germany). The pellet could be stored at −70° C.

1.4 2-DE Gel Electrophoresis

The proteins were separated by a large gel 2-DE technique (gel size 30 cm×23 cm) (28). The isoelectric focusing rod gels (diameter 1.5 mm or 2.5 mm) contained 3.5% acrylamide, 0.3% piperazine diacrylamide (Bio-Rad, Munich, Germany) and a total of 4% w/v carrier ampholytes WITAlytes pH 2-11 (WITA GmbH, Teltow, Germany). About 200 μg to 500 μg of protein were applied to the anodic side of the gel and focused at 8870 Vh. After focusing, the gels were equilibrated for 10 minutes in a buffer containing 125 mM Tris/phosphate, pH 6.8, 40% glycerol, 70 mM dithiothreitol (DTT), and 3% SDS. The equilibrated gels were frozen at −70° C. After thawing, the isoelectric focusing gels were immediately applied to SDS-PAGE gels, which contained 15% w/v acrylamide and 0.2% bisacrylamide. The SDS-PAGE system of Laemmli, 1970 was used, replacing the stacking gel by the equilibrated IEF gel. Electrophoresis was performed using a two-step increase of current, starting with 15 minutes at 120 mA, followed by a run of about 6 hours at 150 mA until the front reached the end of the gel.

1.5 Staining

1.5.1 Staining with Coomassie Blue R-250

Preparative gels were stained with Coomassie Brilliant Blue R-250 (Serva, Heidelberg, Germany). After fixation over night in 1 l 50% ethanol/10% acetic acid/40% water, the gel was stained for at least 5 hours in 1 l 50% methanol/10% acetic acid/40% water, 1 g Coomassie Blue R-250. The staining solution was removed and the gel was destained for 1 hour with 1 l 5% methanol/12.5% acetic acid/82.5% water. Subsequently, the gel was kept for 4 hours in aqueous 7% acetic acid and stored at 4° C. in a plastic foil.

1.5.2 Staining with Silver Nitrate

Analytical gels were stained with silver nitrate. After fixation for at least one hour in 1 l 50% ethanol/10% acetic acid/40% water, the gel was incubated for 2 hours in 1 l 30% ethanol/0.5 M sodium acetate/0.5 glutaraldehyde/0.2% sodium thiosulfate. After washing with water twice for 20 minutes, the gel was stained with 1 l 0.1% silver nitrate/0.01% formaldehyde for 30 minutes. After washing for 30 seconds, the gel was developed for at least 4 minutes in 2.5% sodium carbonate, pH 11.3/0.05 mM sodium thiosulfate/0.01% formaldehyde. The staining process was stopped by applying 0.05 M Titriplex III/0.02% Thimrerosal. The solution was renewed after 15 minutes. Finally, the gels were dried for 3 hours at 70° C. between cellophane membranes using a gel dryer (Model 585, Bio-Rad, München, Germany).

1.6 Tryptic Digestion

The Coomassie Blue R-250 stained single gel spots from Jurkat T-cells were excised with a scalpel and shrunk by addition of 100 μl 50 mM ammonium bicarbonate, pH 7.8/acetonitrile (1:1) for 30 minutes at 37° C. under shaking. Subsequently the solution was exchanged against 100 μl 50 mM ammonium bicarbonate, pH 7.8 for reswelling of the gel piece for 30 minutes at 37° C. under shaking. The gel spots were dried in a vacuum concentrator (Eppendorf, Hamburg, Germany) after removing the buffer. 0.1 μg of trypsin (Promega, Madison, Wis., USA) solved in 1 μl 50 mM acetic acid and 19 μl 50 mM ammonium bicarbonate, pH 7.8 were added. After incubation at 37° C. for 16 hours the supernatant was removed and the gel pieces were washed with 20 μl 0.5% aqueous TFA/acetonitrile (2:1) and again the supernatant was removed. The combined supernatants were evaporated in the vacuum concentrator and solved in 4 μl 0.5% aqueous TFA/acetonitrile (2:1) for the mass spectrometrical analysis.

1.7 Peptide Mass Fingerprinting by MALDI-MS

The mass spectra were recorded by using a time-of-flight delayed extraction MALDI mass spectrometer (Voyager-Elite, Perseptive Biosystems, Framingham, Mass., USA). The samples were mixed in an Eppendorf tube with the same volume of the matrix solution. Twenty mg/ml α-cyano-4-hydroxycinnamic acid (CHCA) in 0.3% aqueous TFA/acetonitrile (1:1) or 50 mg/ml 2,5-dihydroxybenzoic acid (DHB) in 0.3% aqueous TFA/acetonitrile (2:1) were used as matrices. Two μl of the mixtures were applied to a gold-plated sample holder and introduced into the mass spectrometer after drying. The spectra were obtained in the reflectron mode by summing 100-200 laser shots with the acceleration voltage of 20 kV, 70% grid voltage, 0.05 guide wire voltage, 100 ns delay and the low mass gate at 500 m/z.

1.8 Sequencing by ESI-MS/MS

The mass spectra were aquired with a quadrupole/time-of flight ESI mass spectrometer equipped with a nebulized nanoelectrospray Z-spray source (Q-Tof, Micromass, Manchester, GB). Therefore, the tryptic digest was purified with a ZipTip C-18 tip (Millipore, Eschborn, Germany). The sample was evaporated and then dissolved in 2 μl 1% acetic acid/49% water/50% methanol. Subsequently, 1 μl was introduced in the mass spectrometer using a nanospray needle to generate the mass spectra.

1.9 Database Searching

The proteins were identified by using the peptide mass fingerprinting analysis software MS-Fit (http://prospector.ucsf.edu/ucsfhtml3.2/msfit.htm). The NCBI database with the species human and mouse was used for the searches by considering at maximum one missed cleavage site, pyro-Glu formation at N-terminal Gin, oxidation of methionine, acetylation of the N-terminus and modification of cysteines by acrylamide.

The molecular masses and isoelectric points were calculated by employing the software Compute pl/Mw (http://www.expasy.ch/tool/pi_tool.html).

1.10 In Vitro Translation and Cleavage Assay

The cDNAs were translated in vitro using ³⁵S labelled methionine with the T-NT coupled reticulocyte lysate system according to the manufacturer's instructions (Promega, Mannheim, Germany). One μl of the translation product was cleaved with 3 μl active lysate or 20 U caspase-3 (BIOMOL, Hamburg, Germany) in 20 μl cleavage buffer (25 mM Hepes pH 7.5, 1 mM DTT, 1 mM EDTA and the protease inhibitors pefabloc pepstatin, leupeptin and aprotinin) for 1 h at 37° C. For inhibition experiments, 1 μl 5 mM Z-Val-Ala-DL-Asp-fluoromethylketone (Z-VAD-fmk) was added. The cleavage mixture was supplemented with 5 μl loading buffer (1 μl glycerol, 1 μl 10% SDS, 0.25 μl 2-mercaptoethanol, 0.075 mg Tris-base and 0.125 mg bromophenol blue) and applied to a 10% SDS-PAGE gel.

After electrophoresis, the gel was washed, dried and covered with a BioMax MR film (Kodak, Chalon-sur-Saone, France) overnight and then developed.

Active lysate was generated from Jurkat T-cells after 6 h induction of apoptosis with 250 ng/ml alphaCD95 (clone CH11, Immunotech, Marseille, France) and 1 μg/ml cycloheximide. Subsequently, the cells were washed with PBS and incubated for 20 min on ice with lysis buffer (25 mM Hepes, 0.1% Chaps, 1 mM DTT and the protease inhibitors pefabloc, pepstatin, leupeptin and aprotinin).

Afterwards, the cells were homogenized and centrifuged for 5 min at 13.000 U/min (Biofuge fresco, Heraeus Instruments GmbH, Hanau, Germany). The supernatant was aliquoted and stored at −70° C.

In order to either verify or determine the cleavage by caspases, the cDNAs to be tested were cloned and expressed in vitro. The proteins were treated with either a lysate or apoptotic Jurkat T-cells which contained a mixture of active caspases, or with the recombinant purified caspase-3 in the presence or absence of the broad range caspase inhibitor zVAD-fmk. In most cases, the same cleavage pattern was observed for the proteins treated with the active lysate and caspase-3, however, the cleavage by caspase-3 was more efficient.

2. Results for the Total Cell Lysate

2.1 Identification of Apoptosis-Modified Protein Snots

Apoptosis was induced in Jurkat T-cells by treatment with an anti-Fas antibody for six hours. 2-DE gels were produced after lysis of the cells and separation of the proteins. A representative 2-DE gel of Fas-induced Jurkat T-cells is shown in FIG. 1. Approximately 2000 spots were resolved and detected by silver staining. Ten 2-DE gels of apoptotic cells were compared with ten 2-DE gels of Jurkat T-cells (FIG. 2). Protein patterns of apoptosis-induced cells and control cells were found to be highly reproducible. In Fas-induced Jurkat T-cells 24 additional spots and in untreated Jurkat T-cells 21 additional spots were observed. Coomassie stained 2-DE gels were used for the identification by mass spectrometry,

2.2 Identified Proteins

The proteins of the total cell lysate (Table 1a and Table 3) were identified within 21 spots by peptide mass fingerprinting after in-gel digestion with trypsin, elution of the generated peptides and analysis by DE-MALDI-MS (FIGS. 1 and 2). In the total cell lysate, 10 additional proteins were identified after Fas induction, whereas 6 proteins disappeared (Table 3). Four proteins (hnRNP A2/B1, hnRNP C1/C2, p54nrb and Rho GDI 2) were found at different spot positions in negative- and positive Fas cells, whereas the other proteins were only identified at one condition.

The molecular mass of protein spots in 2-DE gels can usually be determined with an accuracy of about 10%. The identified proteins in negative Fas gels displayed the theoretical mass of the corresponding protein. Five of the apoptosis-modified positive Fas proteins showed a significant decreased mass, whereas the remaining three proteins hnRNP C1/C2, p54nrb and splicing factor SRp30c retained the expected theoretical mass. The negative Fas spot of p54nrb showed an increased mass of 3.6 kD in comparison to the positive Fas spot of the same protein (FIG. 3). The negative Fas spot of the hnRNP C1/C2 spots displayed an increased mass of 1 kD and decreased pl of 0.4 in comparison to the positve Fas spots. The mass and pl of the splicing factor SRp30c in Fas-positive Jurkat T-cells showed the theoretical values. These results indicate that predominantly cleavage events have occurred within the identified proteins during the apoptotic process.

The identified protein share similarities concerning function and motifs. The hnRNPs and the splicing factors are involved in the splicing process. 8 proteins contain the RNP-motif and 7 proteins include an aspartic acid/glutamic acid rich domain. Interaction with protein kinase CK2 was already identified for hnRNP A2/B1, hnRNP C1/C2, nucleolin and the transcription factor BTF3.

2.3 Prediction of Cleavage Sites After Fas-Induction

Seven proteins were reduced in mass after Fas-induction. Considering the sequence coverage of the peptide mass fingerprint and the difference of the theoretical and the detected mass and pl lead to calculate approximately the cleavage site of the protein (Table 2). The identified protein spots of hnRNP A2/B1 and Rho GDI 2 was cleaved at the amino-terminal end, hnRNP A1, hnRNP R and p54nrb at the carboxy-terminal end and nucleolin at both sites.

The cleavage sites can be estimated more precisely by taking in account that caspases were responsible for the degradation. These enzymes cleave target proteins at specific aspartic acids. Only one cleavage site is possible for p54nrb, Rho GDI 2 and the amino-terminal cleavage of nucleolin, whereas two sites can be calculated for hnRNP A1, hnRNP A2/B1, hnRNP C1/C2 and for the carboxy-terminal cleavage of nucleolin (Table 2).

Concerning the specificities of the caspases, the most likely cleavage site for hnRNP A2/B1 is the sequence AEVD, for the carboxy-terminal cleavage of nucleolin the sequence AMED. The two possible cleavage sites of hnRNP A1 are quite equal concerning caspase specificity. Two cleavage sites can be calculated for hnRNP C1/C2 but it can be assumed likewise that the known phosphorylation may be the reason for the shift in pl, which is supported by the fact that hnRNP C1/C2 was identified in neighboring seven spots. The possible cleavage of hnRNP R was relatively difficult to calculate. Most reasonable was an amino- and carboxy-terminal cleavage which lead approximately to the found mass and pl.

The RNP consensus sequence of the RNP motif is composed of two short sequences, RNP1 and RNP2, and a number of other conserved amino acids (29). Five of the six identified shortened proteins contain one or more RNP motifs. The RNP1 and RNP2 consensus sequences of hnRNP A1, hnRNP R, p54nrb, one of the two of hnRNP A2/B1 and two of the four of nucleolin are within the sequence of the identified protein spots. No cleavage within the sequence from RNP2 to RNP1 has occurred. On the hand, the carboxy-terminal sequence in hnRNP A1, termed M9, was separated from the protein.

2.4 Results for Cell Compartments

In addition to the total cell lysate, the cytosolic compartment, the nucleus, the mitochondria and the membrane were analysed. Since de novo synthesis of proteins was suppressed, the appearance or disappearance of proteins in cellular compartments after apoptosis induction indicates translocation of these proteins from one compartment to the other (e.g. 60 S ribosomal protein P0, Baf-57, Caf-1, FUSE binding protein 1, GAP SH3 binding protein, HDGF, HnRNP A/B, HnRNP A1, HnRNP A2/B1, HnRNP A3, HnRNP C1/C2, HnRNP D, HnRNP K, KH-type splicing regulatory protein, lamin B1, lamin B2, p54nrb, Rho GDI 2, Tat binding protein 1). After Fas induction, 25 additional proteins could be identified in the cytosol, whereas 12 proteins disappeared (Table 4, FIGS. 3 and 4). In the nucleus, 15 additional proteins could be identified after Fas induction, whereas 37 disappeared (Table 5, FIGS. 5 and 6). In the mitochondria, 10 additional proteins could be identified after Fas induction (Table 6, FIGS. 7 and 8). In the membrane, 22 additional proteins could be identified after Fas induction, whereas 35 disappeared (Table 7, FIGS. 13 and 14). After cis-platin induction, two additional proteins appeared in the total cell lysate, whereas seven proteins disappeared. In the membrane, two additional proteins appeared after apoptosis induction. In the mitochondria, two proteins disappeared (Table 8, FIGS. 15, 16, 17, 18).

3. Discussion

Apoptosis-modified proteins were identified by a proteome approach after Fas-induction. The proteins which were found in the total cell lysate hnRNP A2/B1, hnRNP R, p54nrb, splicing factor ASF-2 and splicing factor SRp30c were not yet described to be related to apoptosis. The five proteins hnRNP A1, hnRNP C1/C2, nucleolin, Rho GDI 2 and transcription factor BTF3 were already known to be associated to apoptosis. These proteins were identified as well by a proteome approach in the human Burkitt Lymphoma cell line HL60 after IgM-mediated apoptosis (18,30,31). However, hnRNP A1, nucleolin and Rho GDI 2 were identified at other spot positions compared to the Jurkat T-cells. These results prove that the proteome approach can be useful to identify apoptosis-modified proteins at different experimental conditions.

Separation of cellular compartments led to a significant increase of the sensitivity of protein detection and identification. In addition the translocation of proteins during apoptosis can be monitored in a highly sensitive way. Protein translocation plays a major role in apoptosis signalling. For example, apoptosis-inducing proteins are released from the mitochondria into the cytosol. Caspase activated DNAse (CAD) translocates from the cytosol to the nucleus. Interference with protein translocation might be a useful approach to modify the apoptosis process. Thus modulating protein translocation offers therapeutic possibilities in both, proliferative diseases with the aim to induce apoptosis as well as degenerative diseases with the aim to prevent apoptosis.

More than 60 substrates for caspases have been already described (6). These proteins can be activated or inactivated due to the cleavage. The caspase substrates are involved in different processes e.g. cell cycle, replication, transcription, translation, DNA cleavage, DNA repair and function as kinases, cytoskeletal and structural proteins. The results of this study indicated that cleavage events have occurred within the identified proteins, probably by caspases.

The most striking feature of the identified apoptosis-modified proteins of the total cell lysate is that eight of the proteins contain the RNP-binding motif and seven of the eight proteins, with the exception of nucleolin, are involved in the splicing process.

The RNP-motif, also known as RBD or RRM (29), was identified in about 300 proteins. It is composed of two consensus sequences, RNP2 and RNP1, and a number of other amino acids within a total length of about 90 amino acids. The three dimensional structure was solved first in the U1A spliceosomal protein. RNA-binding proteins are involved in the regulation of gene expression. In particular, the regulation of RNA by signalling allows a cell to respond much faster to a stimuli than protein expression from de novo transcription. Specific mRNAs can be stored as mRNA-protein complexes and in response to a stimulus the masking proteins are removed or modified and the mRNA is translated. Consideration of the identified protein spots revealed that no cleavage occurred within the RNP-motif. Hence it can be assumed that the RNA-binding properties are probably not affected by the apoptotic process.

Many proteins involved in alternative splicing contain RNA-protein binding motifs. Alternative splicing of pre-mRNA is a process for generating functionally different proteins from the same gene. The splicing reaction is catalyzed by the spliceosome, which is formed by small nuclear ribnucleoproteins (snRNPs) and a large number of splicing factors. In particular, proteins of the SR family play important roles in splicing control. Furhermore, phosphorylation modulates protein-protein interactions within the spliceosome.

An important factor for the complex regulation of apoptosis may well be pre-mRNA splicing. Alternative splicing was identified for some contributors to apoptosis. Death receptors, Bcl-2 family members, caspases and CED-4 showed alternative splice forms (32). Apoptosis-associated proteins can be generated by splicing with different functions and subcellular localization. The potential crucial role in regulation of apoptosis by splicing was confirmed strongly by the fact that the predominantly number and significance of the altered proteins were involved in splicing process.

Reference List

1. Thompson, C. B. (1995) Science 267, 1456-1462

2. Nicholson, D. W. (1996) Nat.Biotechnol. 14, 297-301

3. Cohen, G. M. (1997) Biochem.J. 326, 1-16

4. Thornberry, N. A. and Lazebnik, Y. (1998) Science 281, 1312-1316

5. Miller, L. K. (1999) Trends Cell Biol. 9, 323-328

6. Stroh, C. and Schulze-Osthoff, K. (1998) Cell Death Differ. 5, 997-1000

7. Adams, J. M. and Cory, S. (1998) Science 281, 1322-1326

8. Gross, A., McDonnell, J. M., and Korsmeyer, S. J. (1999) Genes Dev. 13, 1899-1911

9. Ashkenazi, A. and Dixit, V. M. (1999) Curr.Opin.Cell Biol. 11, 255-260

10. Green, D. and Kroemer, G. (1998) Trends Cell Biol. 8, 267-271

11. Green, D. R. and Reed, J. C. (1998) Science 281, 1309-1312

12. Guo, M. and Hay. B. A. (1999) Curr.Opin.Cell Biol. 11, 745-752

13. Downward, J. (1998) Curr.Opin.Genet.Dev. 8, 49-54

14. Griffith, T. S. and Ferguson, T. A. (1997) Immunol.Today 18, 240-244

15. Nagata, S. and Golstein, P. (1995) Science 267, 1449-1456

16. O'Conneli, J., Bennett, M. W., O'Sullivan, G. C., Collins, J. K., and Shanahan, F. (1999) Immunol. Today 20, 46-52

17. Pitti, R. M., Marsters, S. A., Lawrence, D. A., Roy, M., Kischkel, F. C., Dowd, P., Huang, A., Donahue, C. J., Sherwood, S. W., Baldwin, D. T., Godowski, P. J., Wood, W. I., Gurney, A. L., Hillan, K. J., Cohen, R. L., Goddard, A. D., Botstein, D., and Ashkenazi, A. (1998) Nature 396, 699-703

18. Brockstedt, E., Rickers, A., Kostka, S., Laubersheimer, A., Dorken, B., Wittmann-Liebold, B., Bommert, K., and Otto, A. (1998) J.Biol.Chem. 273, 28057-28064

19. Wilkins, M. R., Pasquali, C., Appel, R. D., Ou, K., Golaz, O., Sanchez, J. C., Yan, J. X., Gooley, A. A., Hughes, G., Humphery-Smith, I., Williams, K. L., and Hochstrasser, D. F. (1996) Biotechnology (N.Y.) 14, 61-65

20. O'Farrell, P. H. (1975) J.Biol.Chem. 250, 4007-4021

21. Klose, J. (1975) Humangenetik 26, 231-243

22. Aebersold, R. and Leavitt, J. (1990) Electrophoresis 11, 517-527

23. Fenn, J. B., Mann, M., Meng, C. K., Wong, S. F., and Whitehouse, C. M. (1989) Science 246, 64-71

24. Karas, M. and Hillenkamp, F. (1988) Anal.Chem. 60, 2299-2301

25. Appel, R. D., Bairoch, A., Sanchez, J. C., Vargas, J. R., Golaz, O., Pasquali, C., and Hochstrasser, D. F. (1996) Electrophoresis 17, 540-546

26. Lemkin, P. F. (1997) Electrophoresis 18, 461-470

27. Celis, J. E., Ostergaard, M., Jensen, N. A., Gromova, I., Rasmussen, H. H., and Gromov, P. (1998) FEBS Lett. 430, 64-72

28. Klose, J. and Kobalz, U. (1995) Electrophoresis 16, 1034-1059

29. Burd, C. G. and Dreyfuss, G. (1994) Science 265, 615-621

30. Brockstedt, E., Otto, A., Rickers, A., Bommert, K., and Wittmann-Liebold, B. (1999) J.Protein Chem. 18, 225-231

31. Rickers, A., Brockstedt, E., Mapara, M. Y., Otto, A., Dorken, B., and Bommert, K. (1998) Eur.J.Immunol. 28, 296-304

32. Jiang, Z. H. and Wu, J. Y. (1999) Proc.Soc.Exp.Biol.Med. 220, 64-72

33. Enari M, Sakahira H, Yokoyama H, Okawa K, Iwamatsu A, Nagata S: A caspase-activated DNAse that degrades DNA during apoptosis, and its inhibitor ICAD (1998) Nature 393, 393-396.

34. Yan et al. (1999) J. Biol. Chem. 274, 2145-2156.

TABLE 1a
Table 1a shows several identified apoptosis-modified proteins in Jurkat T-
cells. Apoptosis was induced by Fas.

| | | | Mr | Mr | pI | pI | RNP- | D/E- | |
|---|---|---|---|---|---|---|---|---|---|
| Protein | NCBI | Spot | found | theor. | found | theor. | motif | rich | CK2* |
| hnRNP | 133254 | PF6 | 32100 | 38846 | 8.5 | 9.26 | 2 | − | − |
| A1 | | | | | | | | | |
| hnRNP | 4504447/ | NF4PF4 | 36400 | 36006/ | 9.0 | 8.67/ | 2 | − | + |
| A2/B1 | 133257 | | 29900 | 37429 | 9.3 | 8.97 | | | |
| hnRNP | 133298 | NF1 | 36300 | 33298 | 5.0 | 5.11 | 1 | + | + |
| C1/C2^{#} | | PF1 | 35300 | | 5.4 | | | | |
| hnRNP | 2697103 | PF8 | 49100 | 70943 | 7.3 | 8.23 | 1 | − | − |
| R | | | | | | | | | |
| Nucleolin | 4885511 | PF5 | 18100 | 76344 | 5.2 | 4.59 | 4 | + | + |
| p54nrb | 1895081 | NF3 | 55900 | 54231 | 8.5 | 9.01 | 2 | + | − |
| | | PF3 | 52300 | | 8.1 | | | | |
| Rho | 1707893 | NF2 | 23100 | 22988 | 5.1 | 5.10 | − | + | − |
| GDI 2 | | PF2 | 22100 | | 6.2 | | | | |
| Splicing | 105294 | NF5 | 31400 | 31999 | 5.2 | 5.61 | 1 | + | − |
| factor | | | | | | | | | |
| ASF-2 | | | | | | | | | |
| Splicing | 4506903 | PF7 | 27300 | 25542 | 8.6 | 8.70 | 1 | + | − |
| factor | | | | | | | | | |
| SRp30c | | | | | | | | | |
| Trans- | 29507 | NF6 | 19000 | 17699 | 7.7 | 6.85 | − | + | + |
| cription | | | | | | | | | |
| factor | | | | | | | | | |
| BTF3 | | | | | | | | | |

Known interactions with protein kinase CK2 are displayed with an asterisk *. The sign # indicates that hnRNP C1/C2 was identified in seven spots, three times in negative Fas and four times in positive Fas Jurkat T-cells.

TABLE 1b
Summary of factors modified by apoptosis. Detailed characterization is
given in Tables 2-8 (T = total lysate, M = mitochondria, N = nucleus, C
= cytosol, B = membrane). If nothing else is mentioned, apoptosis was
induced by Fas.

| | | | Modification during | |
|---|---|---|---|---|
| Group | Proteins | Localization | apoptosis | known function |
| hnRNPs | A/B | NC | | RNP motif |
| | A1 | TMNC | | known substrate, RNP |
| | | | | motif |
| | A2/B1 | TMNC | | RNP motif |
| | A3 | N | | RNP motif |
| | C1/C2 | TMN | | known substrate, RNP |
| | | | | motif |
| | D | NC | | RNP motif |
| | F | M | | RNP motif |
| | H | NC | | RNP motif |
| | I | N | | RNP motif |
| | K | NC | | RNP motif |
| | L | N | | RNP motif |
| | R | TN | | RNP motif |
| | JKTBP1 | N | | RNP motif |
| Splicing | SRp30c | TN | only in apoptotic cells; | splicing factor, RNP |
| factors | | | also in the nucleus | motif |
| | P54nrb | TN | modified; processed | splicing factor or |
| | | | | nuclear matrix protein, |
| | | | | respectively, RNP motif |
| | SF2p33 (ASF-2) | TN | missing in the nucleus of | splicing factor; for alter- |
| | | | apoptotic cells | native splicing variant |
| | | | | function in the splicing |
| | | | | of the Caspase-2 was |
| | | | | described, RNP motif |
| | SF SC35 | N | missing in the nucleus of | splicing factor; for alter- |
| | | | apoptotic cells; extreme | native splicing variant |
| | | | IP shift | function in the splicing |
| | | | | of the Caspase-2 was |
| | | | | described, RNP motif |
| | NMP200 (rel. to SF | N | missing in the nucleus of | protein of the nuclear |
| | PRP19) | | apoptotic cells | matrix; unknown |
| | | | | function |
| | PTB-associated SF | C | Cytosol of apoptotic | splicing factor, RNP |
| | | | cells | motif |
| Translation | 60S acidic | MN | ribosomal protein; | known substrate |
| | ribosomal protein | | normal cell in the | |
| | | | nucleus/ER; apoptotic | |
| | | | cells in mitochondria; | |
| | | | altered IP; probably by | |
| | | | phosphorylation | |
| | EF-Tu | N | mitochondrial protein; | translation |
| | | | found in the nucleus of | |
| | | | apoptotic cells | |
| | EF-1 beta | N | missing in the nucleus of | no function known for |
| | | | apoptotic cells | apoptosis |
| | EIF-5A | C | missing in the cytosol of | cellular target of HIV |
| | | | apoptotic cells | type 1 Rev binding |
| | | | | factor |
| Structural | Lamin B1 | NC | nucleus of apoptotic | known substrate |
| | | | cells and as fragment in | |
| | | | the cytosol of apoptotic | |
| | | | cells | |
| | Lamin B2 | NC | nucleus of apoptotic | known substrate |
| | | | cells and cytosol of | |
| | | | apoptotic cells | |
| | Vimentin | N | nucleus of apoptotic | known substrate |
| | | | cells | |
| | Beta-Tubulin | C | cytosol of apoptotic cells | known substrate |
| Signal trans- | GAP SH3 binding | T | Lysate of apoptotic cells, | Ras-Oncogene signal |
| duction | protein | | presumably processed | protein, RNP motif |
| proteins | | | | |
| | Rho GDI2 | TMNC | | known substrate, |
| | | | | published |
| | cGMP-dependent | M | mitochondria of apopto- | Ser/Thr kinase, |
| | protein kinase type I | | tic cells; presumably | unknown function |
| | alpha | | processed | |
| Chromatin | Nucleolin | TC | in the lysate of apoptotic | known substrate; multi- |
| | | | cells, presumably | functional protein, |
| | | | processed | chromatin structure, |
| | | | | RNP motif |
| | Baf-57 | N | missing in the nucleus of | regulator of the chroma- |
| | | | apoptotic cells | tin structure |
| | CAF-1 (RB b.p.) | N | missing in the nucleus of | binds to retinoblastoma, |
| | (WD-repeats) | | apoptotic cells | patent on WD repeat |
| | | | | applications |
| Transcription | BTF3 | TC | missing in the lysate of | already published |
| factor | | | apoptotic cells | |
| | CBF-beta | N | in the nucleus of | binds to enhancers of |
| | | | apoptotic cells | murine leukaemia virus, |
| | | | | Polioma virus, TCR etc., |
| | | | | known alteration in the |
| | | | | case of acute myeloid |
| | | | | leukaemia |
| Proteasome | 26S protease | N | in the nucleus of | regulatory subunit of |
| | subunit 12 | | apoptotic cells | the proteasome |
| | proteasome subunit | C | in the cytosol of | regulatory subunit of |
| | C8 | | apoptotic cells | the proteasome |
| | Tat binding protein- | C | missing in the cytosol of | regulatory subunit of |
| | 1 | | apoptotic cells | the proteasome; HIV |
| | | | | type 1 Tat binding |
| | | | | protein |
| Mitochondrial | Isocitrate | N | in the nucleus of | citrate cycle |
| | dehydrogenase | | apoptotic cells; maybe | |
| | | | shortened | |
| | AOP-1 | N | N-terminal shortened, in | anti-oxidant |
| | | | the nucleus of apoptotic | |
| | | | cells | |
| Miscellaneous | Nucleophosmin | N | missing in apoptotic cells | already published |
| | SYT interacting | N | missing in apoptotic cells | unknown, altered in |
| | protein SIP | | | synovial sarcoma cells |
| | PA1-G | N | in the nucleus of | acetylhydrolase |
| | | | apoptotic cells | |
| | CRHSP-24 | N | in the nucleus of | substrate of calcineurin |
| | | | apoptotic cells | |
| | HCD2 | NC | .in the nucleus of | interaction with amyloid |
| | | | apoptotic cells, missing | β-peptide; Alzheimer's |
| | | | in the cytosol of | disease |
| | | | apoptotic cells, maybe | |
| | | | phosphorylated | |
| | GMP synthase | C | in the cytosol of | synthesis of guanin |
| | | | apoptotic cells | nucleotides, particularly |
| | | | | GTP |
| | FUSE binding | C | in the cytosol of | activation of the far- |
| | protein 1 | | apoptotic cells | upstream element of c- |
| | | | | myc |
| | HDGF | C | missing in the cytosol of | hepatoma-derived |
| | | | apoptotic cells | growth factor, |
| | | | | mitogenic activity for |
| | | | | fibroblasts |

Additional proteins

| | | | Modification during | Known |
|---|---|---|---|---|
| Group | Proteins | Localization | apoptosis | function |
| hnRNPs | HnRNP A0 | B | missing in the membrane | RNP motif |
| | | | of apoptotic cells | |
| | Apobec-1 interacting | B | membrane of apoptotic | RNP motif; interaction |
| | protein | | cells | with apolipoprotein B |
| Splicing | Splicing factor 1 | B | missing in the membrane | KH motif |
| factors | | | of apoptotic cells | |
| | KH-type splicing | C B | missing in the membrane | KH motif |
| | regulatory protein | | of apoptotic cells, | |
| | | | shortened in the cytosol of | |
| | | | apoptotoc cells | |
| Translation | 40 S ribosomal protein | B | membrane of apoptotic | |
| | SA | | cells | |
| | Elongation factor 1- | B | membrane of apoptotic | |
| | delta | | cells | |
| | Elongation initation | B | missing in the membrane | RNP motif |
| | factor 3, subunit 4 | | of apoptotic cells | |
| | Poly(A)-binding | B | missing in the membrane | RNP motif |
| | protein, cytoplasmic 4 | | of apoptotic cells | |
| Structural | Gamma actin | B | missing in the membrane | |
| | | | of apoptotic cells | |
| | Myosin heavy chain | B | membrane of apoptotic | |
| | | | cells | |
| Signal | GAP SH3-binding | B | missing in the membrane | RNP motif |
| transduction | protein 2 | | of apoptotic cells | |
| | Small G protein | C | missing in the cytosol of | Plasma membrane- |
| | | | apoptotic cells | associated GTP binding |
| | | | | protein |
| Chromatin | KIAA1470 | B | membrane of apoptotic | Regulator of chromosome |
| | | | cells | condensation (RCC1)- |
| | | | | motif |
| Mitochondrial | ATP synthase beta chain | B | missing in the membrane | |
| | | | of apoptotic cells | |
| | ATP synthase D chain | B | missing in the membrane | |
| | | | of apoptotic cells | |
| Miscellaneous | Alpha NAC | B | membrane of apoptotic | Nascent-polypeptide- |
| | | | cells | associated complex |
| | | | | protein; transcriptional |
| | | | | coactivator |
| | ARDH | B | membrane of apoptotic | N-terminal |
| | | | cells | acetyltransferase |
| | Cargo selection protein | B | missing in the membrane | Mannose 6-phosphate |
| | | | of apoptotic cells | receptor binding protein |
| | DAZ associated protein | B | missing in th membrane | RNP motif |
| | 1 | | of apoptotic cells | |
| | DEAD box protein | C | cytosol of apoptotic cells | |
| | retinoblastoma | | | |
| | Dihydrofolate reductase | C | missing in the cytosol of | |
| | | | apoptotic cells | |
| | Hydroxyacly-CoA | B | missing in the membrane | Trifunctional protein |
| | dehydrogenase/3- | | of apoptotic cells | |
| | ketoacyl-CoA | | | |
| | thiolase/enoyl-CoA | | | |
| | hydratase | | | |
| | ER-60 | B | missing in the membrane | Disulfide isomerase, |
| | | | of apoptotic cells | thioredoxin domains |
| | HCA56 | B | missing in the membrane | Hepatocellular |
| | | | of apoptoptic cells | carcinoma-associated |
| | | | | antigen |
| | Hsp-105 | C | missing in the cytosol of | Heat shock protein |
| | | | apoptotic cells | |
| | IGF-II mRNA-binding | B | missing in the membrane | RNP motif, KH motif; |
| | protein 1 | | of apoptotic cells | Insulin-like growth factor |
| | | | | mRNA-binding |
| | IGF-II mRNA-binding | B | missing in the membrane | RNP motif, KH motif; |
| | protein 3 | | of apoptotic cells | Insulin-like growth factor |
| | | | | mRNA-binding |
| | Lactate dehydrogenase | B | missing in the membrane | |
| | A | | of apoptotic cells | |
| | NS-associated protein | C B | missing in the membrane | RNP motif |
| | | | of apoptotic cells, | |
| | | | shortened in the cytosol of | |
| | | | apoptotic cells | |
| | RAD 21 | B | membrane of apoptotic | DNA double-strand break |
| | | | cells | repair |
| | RAD 23 homolog B | C | missing in the cytosol of | DNA excision repair |
| | | | apoptotic cells | |
| | T-complex protein 1 | B | membrane of apoptotic | |
| | beta subunit | | cells | |
| | Thioredoxin like protein | B | membrane of apoptotic | |
| | | | cells | |
| | Unnamed protein | C | missing in the cytosol of | |
| | | | apoptotic cells | |

T = Total lysate
M = Mitochondria
N = Nucleus
C = Cytosol
B = Membrane

Additional proteins, cis-platin induced

| | | | Modification during | |
|---|---|---|---|---|
| Group | Proteins | Localization | apoptosis | Known function |
| hnRNPs | HnRNP E1 | TL | missing in the lysate | KH-motiv |
| | | | of apoptotic cells | |
| | HnRNP M | TL | missing in the lysate | RNP-motiv |
| | | | of apoptotic cells | |
| Structural | Alpha-Fodrin | TL | in the lysate of apop. cells | |
| Proteasome | 26 S protease subunit 4 | M | mitochondria of apop- | |
| | | | totic cells | |
| | Proteasome subunit | M | mitochondria of apop- | |
| | alpha type 1 | | totic cells | |
| Miscellaneous | Chondrosarcoma- | TL | missing in the lysate | |
| | associated protein 2 | | of apoptotic cells | |
| | ELAV-like 1 (Hu | TL | missing in the lysate | RNP-motiv |
| | antigen R) | | of apoptotic cells | |
| | Glutathion S- | TL | missing in the lysate | |
| | transferase | | of apoptotic cells | |
| | Hsp-60 | TL | in the lysate of apop. cells | Chaperone |
| | Mortalin-2 (Heat shock | B | membrane of apopototic | Chaperone |
| | 70kd protein 9B) | | cells | |
| | Prohibitin | B | membrane of apopototic | Inhibitor of DNA |
| | | | cells | synthesis |
| | SKI interacting protein | TL | missing in the lysate | |
| | | | of apoptotic cells | |
| | VDAC 3 | TL | missing in the lysate | Ion channel |
| | | | of apoptotic cells | |

TL = Total lysate
B = Membrane
M = Mitochondria

TABLE 2
Prediction of cleavage sites for apoptosis-modified proteins found in the
total cell lysate

| | | | | | | | | | Puta- |
|---|---|---|---|---|---|---|---|---|---|
| | | | | | | | | | tive |
| | | | | | | | | | cleaved |
| | No. | Sequence | Cleavage | Start-end | Mass | Mass | | | se- |
| Protein | AA | coverage | site | AA | (kDa) | found | pI | pI found | quence |
| hnRNP | 371 | 15-178 | CT | 1-288 | 30.5 | 32.1 | 8.4 | 8.5 | GSYD |
| A1 | | | | 1-314 | 32.9 | | 8.4 | | SYND |
| hnRNP | 351 | 102-350 | NT* | 49-353 | 31.6 | 29.9 | 8.9 | 9.3 | KLTD |
| A2/B1 | | | | 56-353 | 30.8 | | 9.2 | | VMRD |
| | | | | 76-353 | 28.6 | | 8.8 | | AEVD |
| HnRNP | 303 | 18-151 | — | 1-295 | 32.5 | 35.3 | 5.2 | 5.4 | EGED |
| C1/C2 | | | | 10-303 | 32.3 | | 5.0 | | NKTD |
| hnRNP | 624 | 134-441 | CT | 1-463 | 52.1 | 49.1 | 5.9 | 7.3 | YPPD |
| R | | | | 20-463 | 49.9 | | 6.5 | | EPMD + |
| | | | | | | | | | YPPD |
| Nucleo- | 706 | 458-624 | NT + CT | 454-628 | 19.4 | 18.1 | 5.0 | 5.2 | TEID + |
| lin | | | | 454-632 | 19.8 | | 4.9 | | AMED |
| | | | | | | | | | TEID + |
| | | | | | | | | | GEID |
| p54nrb | 471 | 76-336 | (CT*) | 1-422 | 49.2 | 52.3 | 8.4 | 8.1 | MMPD |
| Rho | 201 | 22-196 | NT | 22-196 | 20.9 | 22.1 | 6.2 | 6.2 | DELD |
| GDI 2 | | | | | | | | | |

The asterisk * displays that the comparison of the PMF of negative- and positive Fas showed an additional intense peak of the negative Fas spot outside the covered sequence and confirms the cleavage site (FIG. 3). In parenthesis means that the cleavage site could not clearly identified only by sequence coverage of the PMF of the positive Fas spot.

TABLE 3
Table 3 shows proteins of the total cell lysate. Apoptosis was induced by Fas.

| | | | Mr | Mr | pI | pI |
|---|---|---|---|---|---|---|
| Spot | Protein | NCBI | theor. | found | theor. | found |
| PF1 | hnRNP C1/C2 | 4758544 | 31966 | 35300 | 5.10 | 5.3 |
| PF2 | RhoGDI 2 | 1707893 | 22988 | 22400 | 5.10 | 6.2 |
| PF3 | P54nrb | 1895081 | 54231 | 52300 | 9.01 | 8.1 |
| PF4 | hnRNP A2/B1 | 4504447/ | 36006/ | 36300 | 8.67/8.97 | 9.6 |
| | | 133257 | 37429 | | | |
| PF5 | Nucleolin | 4885511 | 76344 | 18100 | 4.59 | 5.2 |
| PF6 | hnRNP A1 | 133254 | 38846 | 35200 | 9.26 | 9.6 |
| PF7 | Splicing factor SRp30c | 4506903 | 25542 | 27300 | 8.70 | 8.6 |
| PF8 | hnRNP R | 2697103 | 70943 | 49100 | 8.23 | 7.3 |
| PF9 | Unknown¹ | = | = | 24900 | = | 5.3 |
| PF10 | GAP SH3 binding protein | 5031703 | 52164 | 37000 | 5.37 | 6.2 |
| NF1 | hnRNP C1/C2 | 4758544 | 31966 | 36300 | 5.10 | 5.3 |
| NF2 | RhoGDI 2 | 1707893 | 22988 | 22400 | 5.10 | 6.4 |
| NF3 | P54nrb | 1895081 | 54231 | 55900 | 9.01 | 8.5 |
| NF4 | hnRNP A2/B1 | 4504447/ | 36006/ | 35700 | 8.67/8.97 | 8.7 |
| | | 133257 | 37429 | | | |
| NF5 | Splicing fact r 2p33 (ASF-2) | 105294 | 31999 | 31400 | 5.61 | 5.2 |
| NF6 | Transcription factor BTF3 | 29507 | 17699 | 19000 | 6.85 | 7.7 |

¹Peptide mass fingerprint of the tryptic digesti n (internal name: PFC6D)

TABLE 4
Table 4 shows proteins of the cytosol. Apoptosis was induced by Fas.

| | | | Mr | Mr | pI | pI |
|---|---|---|---|---|---|---|
| Spot | Protein | NCBI | theor. | found | theor. | found |
| Cpf1 | Beta-Tubulin | 135448 | 49759 | 49800 | 4.75 | 5.2 |
| Cpf2 | PTB-associated splicing factor | 4826998 | 76149 | 89000 | 9.45 | 8.7 |
| Cpf3 | PTB-associated splicing factor | 4826998 | 76149 | 76000 | 9.45 | 8.3 |
| Cpf4 | GMP synthase | 4504035 | 76715 | 62100 | 6.42 | 6.9 |
| Cpf5 | FUSE binding protein 1 | 4503801 | 67534 | 65200 | 7.21 | 7.7 |
| Cpf6 | FUSE binding protein 1 | 4503801 | 67534 | 65400 | 7.21 | 7.8 |
| Cpf7 | hnRNP D | 870749 | 38434 | 43100 | 7.61 | 7.4 |
| Cpf8 | hnRNP A/B | 4758542 | 31233 | 39100 | 9.35 | 6.6 |
| Cpf9 | hnRNP A/B | 4758542 | 31233 | 36200 | 9.35 | 7.6 |
| Cpf10 | hnRNP A2/B1 | 4504447/ | 36006/ | 39000 | 8.67/8.97 | 9.6 |
| | | 133257 | 37429 | | | |
| Cpf11 | hnRNP A2/B1 | 4504447/ | 36006/ | 35700 | 8.67/8.97 | 8.4 |
| | | 133257 | 37429 | | | |
| Cpf12 | hnRNP A1 | 133254 | 38846 | 35200 | 9.26 | 9.5 |
| Cpf13 | hnRNP A1 | 133254 | 38846 | 35200 | 9.26 | 9.6 |
| Cpf14 | Proteasome subunit C8 | 130859 | 28433 | 26600 | 5.19 | 5.2 |
| Cpf15 | Lamin B2 | 547822 | 59001 | 22400 | 5.87 | 5.8 |
| Cpf16 | Nucleolin | 4885511 | 76344 | 19000 | 4.59 | 5.2 |
| Cpf17 | Lamin B1 | 5031877 | 66408 | 22400 | 5.11 | 5.0 |
| Cpf18 | RhoGDI 2 | 1707893 | 22988 | 22400 | 5.10 | 6.2 |
| Cpf19 | RhoGDI 2 | 1707893 | 22988 | 22400 | 5.10 | 6.4 |
| Cpf20 | hnRNP A1 | 133254 | 38846 | 32100 | 9.26 | 8.1 |
| Cpf21 | hnRNP A1 | 133254 | 38846 | 35200 | 9.26 | 9.6 |
| Cnf1 | hnRNP K | 631471 | 51072 | 65500 | 5.14 | 5.2 |
| Cnf2 | hnRNP H | 1710632 | 49229 | 54100 | 5.89 | 6.1 |
| Cnf3 | HDGF | 4758516 | 26788 | 36100 | 4.70 | 4.7 |
| Cuf4 | Tat binding protein-1 | 4506211 | 45165 | 45100 | 5.31 | 5.0 |
| Cnf5 | RhoGDI 2 | 1707893 | 22988 | 23100 | 5.10 | 5.1 |
| Cnf6 | EIF-5A | 4503545 | 16701 | 17000 | 5.08 | 5.3 |
| Cnf7 | Transcription factor BTF3 | 29507 | 17699 | 19000 | 6.85 | 7.7 |
| Cnf8 | HCD2 | 4758504 | 26923 | 24800 | 7.65 | 6.2 |

Proteins of the cytosol

| | | | Mr | Mr | pI | pI |
|---|---|---|---|---|---|---|
| Spot | Protein | NCBI | theor. | found | theor. | found |
| Cpf22 | DEAD box protein retinoblastoma | 4826686 | 82410 | 75200 | 6.8 | 7.0 |
| Cpf23 | KH-type splicing regulatory protein | 4504865 | 73140 | 72000 | 6.9 | 6.9 |
| Cpf24 | NS1-associated protein 1 | 5453806 | 62640 | 54100 | 6.9 | 6.1 |
| Cpf25 | NS1-associated protein 1 | 5453806 | 62640 | 54100 | 6.9 | 6.1 |
| Cnf9 | Hsp-105 | 5729879 | 92100 | 89500 | 5.3 | 5.3 |
| Cnf10 | Unnamed protein | 7020309 | 59330 | 88700 | 6.1 | 6.6 |
| Cnf11 | RAD23 homolog B | 4506387 | 43150 | 61400 | 4.8 | 4.8 |
| Cnf12 | Dihydrofolate reductase | 229860 | 21321 | 21800 | 7.02 | 7.4 |
| | Small G-protein | 4506381 | 21450 | | 8.77 | |

TABLE 5
Table 5 shows proteins of the nucleus. Apoptosis was induced by Fas.

| | | | Mr | Mr | pI | pI |
|---|---|---|---|---|---|---|
| Spot | Protein | NCBI | theor. | found | theor. | found |
| Kpf1 | hnRNP R | 2697103 | 70943 | 49100 | 8.23 | 7.2 |
| Kpf2 | Isocitrate dehydrogenase | 4504575 | 46644 | 43100 | 8.32 | 8.2 |
| Kpf3 | Elongation factor Tu | 4507733 | 49540 | 41400 | 7.26 | 7.0 |
| Kpf4 | 26S proteasome regulatory chain 12 | 4506231 | 37060 | 38800 | 6.11 | 7.2 |
| Kpf5 | hnRNP C1/C2 | 4758544 | 31966 | 35300 | 5.10 | 5.3 |
| Kpf6 | hnRNP A2/B1 | 4504447/ | 36006/ | 30000 | 8.67/ | 9.0 |
| | | 133257 | 37429 | | 8.97 | |
| Kpf7 | Splicing factor SRp30c | 4506903 | 25542 | 28300 | 8.74 | 8.6 |
| Kpf8 | PA1-G | 4505587 | 25734 | 28500 | 6.33 | 6.3 |
| Kpf9 | HCD2 | 4758504 | 26923 | 24800 | 7.65 | 6.2 |
| Kpf10 | AOP-1 | 5802974 | 27692 | 22500 | 7.67 | 6.1 |
| Kpf11 | Rho GDI 2 | 1707893 | 22988 | 22400 | 5.10 | 6.2 |
| Kpf12 | Rho GDI 2 | 1707893 | 22988 | 22400 | 5.10 | 6.4 |
| Kpf13 | Rho GDI 2 | 1707893 | 22988 | 22100 | 5.10 | 6.4 |
| | | 2498753 | 21508 | | | |
| | CBF-beta | | | | 6.23 | |
| Kpf14 | CRHSP-24 | 4583307 | 15934 | 21300 | 8.41 | 8.8 |
| Kpf15 | Unknown² | — | — | 59100 | — | 8.3 |
| Knf1 | hnRNP K | 631471 | 51072 | 65600 | 5.14 | 5.2 |
| Knf2 | Lamin B1 | 125953 | 66408 | 65600 | 5.11 | 5.2 |
| Knf3 | hnRNP K | 631471 | 51072 | 65100 | 5.14 | 5.4 |
| Knf4 | Lamin B2 | 547822 | 59001 | 62800 | 5.87 | 5.4 |
| Knf5 | hnRNP K | 631471 | 51072 | 59700 | 5.14 | 6.0 |
| Knf6 | NMP200 (related to splicing factor | 5689738 | 55181 | 55500 | 6.14 | 6.4 |
| | PRP19) | | | | | |
| Knf7 | BAF57 | 4507089 | 46649 | 54700 | 4.85 | 4.9 |
| Knf8 | Vimentin | 2119204 | 53651 | 56200 | 5.06 | 5.1 |
| Knf9 | CAF-1 | 422892 | 46158 | 53000 | 4.90 | 4.7 |
| Knf10 | hnRNP H | 1710632 | 49229 | 50600 | 5.89 | 6.1 |
| Knf11 | Splicing factor 2p33 (ASF-2) | 105294 | 31999 | 31400 | 5.61 | 5.2 |
| Knf12 | hnRNP H | 1710632 | 49229 | 42100 | 5.89 | 6.6 |
| Knf13 | Splicing factor 2p33 (ASF-2) | 105294 | 31999 | 31400 | 5.61 | 5.1 |
| Knf14 | hnRNP A/B | 4758542 | 31233 | 39000 | 9.35 | 6.4 |
| Knf15 | hnRNP C1/C2 | 4758544 | 31966 | 36300 | 5.10 | 5.0 |
| Knf16 | Nucleophosmin | 114762 | 32575 | 35300 | 4.64 | 4.8 |
| Knf17 | 60S acidic ribosomal protein | 4506667 | 34273 | 33500 | 5.72 | 5.8 |
| Knf18 | JKTBP1 | 2780748 | 33589 | 36100 | 6.85 | 6.3 |
| Knf19 | JKTBP1 | 2780748 | 33589 | 36100 | 6.85 | 6.6 |
| Knf20 | SYT interacting protein SIP | 5454064 | 69492 | 73000 | 9.68 | 9.0 |
| Knf21 | hnRNP L | 4557645 | 60187 | 67400 | 6.65 | 7.4 |
| Knf22 | hnRNP I | 131528 | 57221 | 53700 | 9.22 | 8.5 |
| Knf23 | hnRNP I | 131528 | 57221 | 53900 | 9.22 | 8.6 |
| Knf24 | P54nrb | 1895081 | 54231 | 54000 | 9.01 | 9.2 |
| Knf25 | hnRNP D | 870749 | 38434 | 44100 | 7.61 | 6.9 |
| Knf26 | hnRNP A1 | 133254 | 38846 | 35200 | 9.26 | 9.6 |
| Knf27 | hnRNP A2/B1 | 4504447/ | 36006/ | 35700 | 8.67/ | 8.4 |
| | | 133257 | 37429 | | 8.97 | |
| Knf28 | hnRNP A3 | 1710627 | 39686 | 39000 | 8.74 | 9.6 |
| Knf29 | hnRNP A2/B1 | 4504447/ | 36006/ | 36400 | 8.67/ | 9.7 |
| | | 133257 | 37429 | | 8.97 | |
| Knf30 | hnRNP A3 | 1710627 | 39686 | 36400 | 8.74 | 8.3 |
| Knf31 | hnRNP A2/B1 | 4504447/ | 36006/ | 36200 | 8.67/ | 8.9 |
| | | 133257 | 37429 | | 8.97 | |
| Knf32 | hnRNP A2/B1 | 4504447/ | 36006/ | 35700 | 8.67/ | 8.2 |
| | | 133257 | 37429 | | 8.97 | |
| Knf33 | Splicing factor SC35 | 539663 | 25476 | 28100 | 11.86 | 5.1 |
| Knf34 | RhoGDI 2 | 1707893 | 22988 | 23100 | 5.10 | 5.1 |
| Knf35 | RhoGDI 2 | 1707893 | 22988 | 21300 | 5.10 | 4.8 |
| Knf36 | Elongation factor 1-beta | 4503477 | 24763 | 24800 | 4.50 | 4.5 |
| Knf37 | Unknown¹ | — | — | 61300 | — | 6.6 |

¹Peptide mass fingerprint of the tryptic digestion and MS/MS-spectrum of mass 1649,76 dalton with the sequence TPGT(F/Mox)E (internal name: KNFE3)
²Peptide mass fingerprint of the tryptic digestion (internal name: KPF₁)

TABLE 6
Table 6 shows proteins of the mitochondria. Apoptosis was induced by Fas.

| | | | Mr | Mr | pI | pI |
|---|---|---|---|---|---|---|
| Spot | Protein | NCBI | theor. | found | theor. | found |
| Mpf1 | hnRNP F | 4836760 | 45672 | 47600 | 5.38 | 5.2 |
| Mpf2 | hnRNP C1/C2 | 4758544 | 31966 | 35300 | 5.10 | 5.3 |
| Mpf3 | CGMP-dependent protein kinase type I | 6225588 | 76364 | 45300 | 5.74 | 6.2 |
| | alpha | 4504453 | 51072 | | 5.14 | |
| | hnRNP K | | | | | |
| Mpf4 | 60S acidic ribosomal protein | 4506667 | 34273 | 33300 | 5.72 | 6.1 |
| Mpf5 | hnRNP A2/B1 | 4504447/ | 36006/ | 36300 | 8.67/8.97 | 9.6 |
| | | 133257 | 37429 | | | |
| Mpf6 | hnRNP A2/B1 | 4504447/ | 36006/ | 35700 | 8.67/8.97 | 8.7 |
| | | 133257 | 37429 | | | |
| Mpf7 | hnRNP A1 | 133254 | 38846 | 35200 | 9.26 | 9.6 |
| Mpf8 | hnRNP A1 | 133254 | 38846 | 35200 | 9.26 | 9.7 |
| Mpf9 | RhoGDI 2 | 1707893 | 22988 | 22100 | 5.10 | 6.2 |
| Mpf10 | RhoGDI 2 | 1707893 | 22988 | 22100 | 5.10 | 6.4 |

TABLE 7
Table 7 shows proteins of the membrane. Apoptosis was induced by Fas.

| | | | Mr | Mr | pI | pI |
|---|---|---|---|---|---|---|
| Spot | Protein | NCBI | theor. | found | theor. | found |
| Bpf1 | PTB associated splicing factor | 4826998 | 76150 | 89200 | 9.5 | 8.3 |
| Bpf2 | Myosin heavy chain, nonmuscle | 189036 | 145080 | 78500 | 5.2 | 5.4 |
| Bpf3 | Rad 21 | 1620398 | 71670 | 70200 | 4.5 | 4.9 |
| Bpf4 | Fuse binding protein 1 | 4503801 | 67530 | 65400 | 7.2 | 7.8 |
| Bpf5 | Caf-1 | 422892 | 46160 | 53000 | 4.9 | 4.7 |
| Bpf6 | Baf-57 | 4507089 | 46650 | 54700 | 4.9 | 4.9 |
| | Beta Tubulin | 135448 | 49760 | | 4.7 | |
| Bpf7 | 40 S ribosomal protein SA | 86715 | 31780 | 43000 | 4.8 | 4.8 |
| Bpf8 | Tat binding protein 1 | 4506211 | 45150 | 42900 | 5.3 | 5.4 |
| Bpf9 | KIAA1470 | 7959201 | 60460 | 46300 | 9.4 | 9.2 |
| Bpf10 | Apobec-1 interacting protein | 1814274 | 36590 | 38600 | 9.1 | 7.7 |
| Bpf11 | Gap SH3 binding protein | 5031703 | 52150 | 36300 | 5.4 | 6.2 |
| Bpf12 | HnRNP C1/C2 | 4758544 | 31950 | 35300 | 5.1 | 5.3 |
| Bpf13 | HDGF | 4758516 | 26770 | 36100 | 4.7 | 4.7 |
| Bpf14 | EF-1 delta | 461994 | 31220 | 31800 | 5.0 | 5.0 |
| | Thioredoxin-like protein | 4759274 | 32230 | | 4.8 | |
| Bpf15 | ARDH | 728880 | 26440 | 24700 | 5.4 | 5.8 |
| Bpf16 | Alpha NAC | 5031931 | 23370 | 26800 | 4.5 | 5.0 |
| Bpf17 | Alpha NAC | 5031931 | 23370 | 26600 | 4.5 | 5.2 |
| Bpf18 | Alpha NAC | 5031931 | 23370 | 26600 | 4.5 | 5.2 |
| Bpf19 | HnRNP A2/B1 | 4504447/ | 36000/ | 32400 | 8.7/ | 9.5 |
| | | 133257 | 37430 | | 8.9 | |
| Bpf20 | T-complex protein 1 beta subunit | 1871210 | 22920 | 26700 | 5.9 | 6.7 |
| Bpf21 | RhoGDI 2 | 1707893 | 22970 | 22300 | 5.1 | 6.4 |
| Bpf22 | RhoGDI 2 | 1707893 | 22970 | 22300 | 5.1 | 6.2 |
| Bnf1 | KH-type splicing regulatory protein | 4504865 | 73140 | 78900 | 6.9 | 6.4 |
| Bnf2 | KH-type splicing regulatory protein | 4504865 | 73140 | 78900 | 6.9 | 6.5 |
| Bnf3 | KH-type splicing regulatory protein | 4504865 | 73140 | 76600 | 6.9 | 6.4 |
| Bnf4 | FUSE binding protein 1 | 4503801 | 67530 | 70900 | 7.2 | 6.5 |
| Bnf5 | FUSE binding protein 1 | 4503801 | 67530 | 70600 | 7.2 | 6.7 |
| | GAP SH3 binding protein 2 | 3098601 | 50750 | | 5.3 | |
| Bnf6 | Splicing factor 1 | 1620403 | 68630 | 76000 | 9.3 | 7.5 |
| Bnf7 | HCA56 | 7678701 | 64730 | 75800 | 7.8 | 7.8 |
| Bnf8 | IGF-II mRNA-binding protein 3 | 4191612 | 63690 | 69100 | 9.2 | 8.2 |
| Bnf9 | Hydroxyacyl-CoA dehydrogenase/3-kecoacyl- | 4504325 | 82960 | 70600 | 9.2 | 8.9 |
| | CoA thiolase/enoyl-CoA hydratase | | | | | |
| Bnf10 | Poly(A)-binding protein cytoplasmic 4 | 4504715 | 70760 | 70600 | 9.5 | 9.2 |
| Bnf11 | IGF-II mRNA-binding protein 1 | 5729882 | 63460 | 65700 | 9.5 | 9.2 |
| Bnf12 | IGF-II mRNA-binding protein 1 | 5729882 | 63460 | 65700 | 9.5 | 9.1 |
| Bnf13 | IGF-II mRNA-binding protein 3 | 4191612 | 63690 | 68900 | 9.2 | 8.5 |
| Bnf14 | NS-associated protein 1 | 5453806 | 62640 | 67900 | 6.9 | 7.6 |
| Bnf15 | Gap SH3 binding protein | 5031703 | 52150 | 70000 | 5.4 | 5.4 |
| Bnf16 | Gap SH3 binding protein | 5031703 | 52150 | 70000 | 5.4 | 5.5 |
| Bnf17 | HnRNP K | 4504453 | 51050 | 65100 | 5.1 | 5.4 |
| Bnf18 | ATP synthase beta chain | 114549 | 56640 | 56600 | 5.3 | 5.0 |
| Bnf19 | ER-60 | 4885359 | 56770 | 54100 | 5.9 | 6.1 |
| Bnf20 | Tat binding protein 1 | 4506211 | 45150 | 46200 | 5.3 | 5.1 |
| Bnf21 | Cargo selection protein | 8134735 | 47030 | 40000 | 5.3 | 5.1 |
| | Gamma-actin | 7441428 | 41790 | | 5.3 | |
| Bnf22 | Elongation initiation factor 3, subunit 4 | 2460200 | 35.590 | 46.200 | 5.9 | 6.1 |
| Bnf23 | Hn RNP I | 131528 | 57200 | 53700 | 9.3 | 8.5 |
| Bnf24 | Hn RNP I | 131528 | 57200 | 53900 | 9.3 | 8.7 |
| Bnf25 | Hn RNP I | 131528 | 57200 | 53700 | 9.3 | 9.0 |
| Bnf26 | Hn RNP I | 131528 | 57200 | 53700 | 9.3 | 9.1 |
| Bnf27 | Hn RNP I | 131528 | 57200 | 53000 | 9.3 | 9.4 |
| Bnf28 | DAZ associated protein 1 | 9506537 | 43410 | 48200 | 9.0 | 8.1 |
| Bnf29 | Elongation initiation factor 3, subunit 4 | 2460200 | 35590 | 41200 | 5.9 | 6.2 |
| Bnf30 | HnRNP C1/C2 | 4758544 | 31950 | 35300 | 5.1 | 5.3 |
| Bnf31 | HnRNP A0 | 8134660 | 30840 | 35600 | 9.3 | 9.9 |
| Bnf32 | Lactate dehydrogenase A | 5031857 | 36690 | 35400 | 8.4 | 8.2 |
| Bnf33 | RhoGDI 2 | 1707893 | 22990 | 23100 | 5.1 | 5.1 |
| Bnf34 | ATP synthase D chain | 6831494 | 18360 | 23000 | 5.2 | 5.2 |
| Bnf35 | TF BTF3 | 29507 | 17680 | 19000 | 6.8 | 7.7 |

TABLE 8
Table 8 shows proteins of the total cell lysate,
the membrane and the mitochondrial fraction.
Apoptosis was induced by Fas.

| | | | Mr | Mr | pI | pI |
|---|---|---|---|---|---|---|
| Spot | Protein | NCBI | theor. | found | theor. | found |

Proteins of the total lysate, cis-platin induced

| | | | | | | |
|---|---|---|---|---|---|---|
| PP1 | Alpha-Fodrin | 4507191 | 284.26 | 84.50 | 5.2 | 5.7 |
| PP2 | Hsp-60 | 306890 | 61.04 | 45.90 | 5.7 | 6.0 |
| NP1 | Chondrosarcoma-associated protein 2 | 5901878 | 65.57 | 78.90 | 6.3 | 6.5 |
| NP2 | ELAV-like 1 (Hu antigen R) | 4503551 | 36.04 | 35.30 | 9.4 | 9.4 |
| NP3 | HnRNP M | 5174611 | 59.95 | 63.30 | 9.0 | 7.9 |
| NP4 | HnRNP EI | 1215671 | 37.48 | 41.00 | 6.7 | 6.9 |
| NP5 | SKI interacting protein | 6912675 | 61.49 | 64.00 | 9.5 | 9.5 |
| NP6 | Glutathione S-transferase | 31948 | 23.21 | 23.00 | 5.4 | 5.7 |
| NP7 | VDAC 3 | 5032221 | 30.64 | 33.40 | 9.2 | 8.8 |

Proteins of the membrane, cis-platin induced

| | | | | | | |
|---|---|---|---|---|---|---|
| NP1 | Mortalin-2 (Heat shock 70kd protein 9B) | 4758570 | 73.78 | 69.60 | 5.97 | 5.40 |
| NP2 | Prohibitin | 4505773 | 29.80 | 26.20 | 5.57 | 5.60 |

Proteins of the mitochondrion, cis-platin induced

| | | | | | | |
|---|---|---|---|---|---|---|
| NP1 | 26S protease regulatory subunit 4 | 345717 | 49.24 | 56.80 | 5.77 | 6.00 |
| NP2 | Proteasome subunit alpha type 1 | 13543551 | 29.58 | 32.50 | 6.15 | 6.20 |

TABLE 9
Caspase cleavage sites (see also Table 2)

| | |
|---|---|
| G3BP | 164 EVVPDDSGT 172 |
| G3BP | 418 AREGDRRDN 426 |
| human 1A cAbI | 526 PELPTKTRTSRRAAEHRDTTD- |
| | VPEMPHSKGQGESD 560 |
| human 1A cAbI | 650 PLDTADPAKSP 660 |
| human 1A cAbI | 934 ATSLVDAVNSD 944 |
| mouse I cAbI | 526 PELPTKTRTCRRAAEQKDAPD- |
| | TPELLHTKGLGESD 560 |
| vAbI | 647 PELPTKTRTCRRAAEQKASPPS- |
| | LTPKLLRRQVTASPS 683 |
| p54rn | 224 EPMDQLDDEEGLP 236 |
| p54rn | 276 EMEKQQQDQVDRNIK 290 |
| p54rn | 412 APPGPATMMPDGTLGLTP 429 |

GSYD
SYND
KLTD
VMRD
AEVD
EGED
NKTD
YPPD
EPMD
TEID
AMED
GEID
MMPD
DELD

1. An apoptosis-associated and/or -modified protein selected from GAP SH3 binding protein, HCD2 and AOP-1 or proteolytic fragments thereof.

2. Use of a protein of claim 1 as target for the diagnosis, prevention or treatment of apoptosis-associated diseases.

3. The use of claim 2 for the manufacture of a pharmaceutical agent.

4. Use of a protein of claim 1 in a method for identifying apoptosis modulators.

5. A method for characterizing and/or identifying apoptosis-modified proteins comprising the steps:
(a) providing a first extract and a second extract comprising soluble proteins, wherein said first extract is from a cell without apoptosis induction and said second extract is from a cell after apoptosis induction,
(b) separating said first and second extracts by two-dimensional gel electrophoresis, wherein first and second proteome patterns each comprising a plurality of protein species are obtained,
(c) comparing said first and second proteome patterns and
(d) characterizing and/or identifying apoptosis-modified protein species.

6. The method of claim 5, wherein after apoptosis induction substantially no synthesis of new proteins has been allowed.

7. The method of claim 6, wherein the protein biosynthesis has been substantially blocked by an inhibitor.

8. The method of claim 6 or 7, wherein apoptosis induction has been carried out for a period of time which is too short to allow a substantial synthesis of new proteins.

9. The method of any one of claims 5-8, wherein said two-dimensional gel electrophoresis comprises (i) separation in a first dimension according to the isoelectric point and (ii) separation in a second dimension according to size.

10. The method of any one of claims 5-9, wherein the apoptosis-modified protein species are selected from protein species which (i) are located at different positions on the two-dimensional gels from the first and second extracts and/or (ii) have a different intensity on the two-dimensional gels from the first and second extracts.

11. The method of any one of claims 5-10, wherein the protein species are characterized by peptide fingerprinting.

12. The method of claim 11, wherein the peptides are characterized by mass spectrometry and/or at least partial sequencing.

13. The method of any one of claims 5-12, wherein said cell is a mammalian cell.

14. The method of claim 13, wherein said cell is a human cell.

15. The method of claim 13 or 14, wherein said cell is a T-cell.

16. The method of claim 15, wherein said T-cell is the T-cell line Jurkat E6 (ATCC TIB 152).

17. The method of any one of claims 5-16, wherein the apoptosis is induced by an anti-Fas antibody or by treatment with cis-platin.

18. The method of any one of claims 5-17, wherein the apoptosis-modified protein species are selected from heterogeneous nuclear ribonucleoproteins, splicing factors, translation factors, structural proteins, signal transduction proteins, chromatin associated proteins, transcription factors, proteasome subunits, mitochondrial proteins, nucleophosmin, SYT interacting protein SIP, PA1-G, CRHSP-24, HCD2, GMP synthase, FUSE binding protein 1, HDGF, PFC6D, KPF1, KNFE3 having the partial sequence TPGT (F/Mox)E, alpha NAC, ARDH, cargo selection protein, DAZ associated protein 1, DEAD box protein retinoblastoma, dihydrofolate reductase, hydroxyacyl-CoA dehydrogenase/3-ketoacyl-CoA thiolase/enoyl-CoA hydratase, ER-60, HCA56, Hsp-105, IGF-II mRNA-binding protein 1, IGF-II mRNA-binding protein 3, lactate dehydrogenase A, NS-associated protein, RAD 21, RAD 23 homolog B, T-complex protein 1 beta subunit, thioredoxin like protein, an unnamed protein (NCBI 7020309), and c-Abl or a partial sequence derived therefrom by substitution and/or deletion of one or more amino acids.

19. The method of any one of claims 5-18 further comprising
(e) determining if the apoptosis-modified proteins are present in subjects suffering from apoptosis-associated diseases.

20. Proteome from an apoptotic T-cell or a compartment thereof consisting of a pattern of individual proteins obtainable by the method of any one of claims 5-19.

21. The proteome of claim 20 containing the proteins as shown in Table 1 or at least a part thereof.

22. Apoptosis-associated and/or -modified protein selected from heterogeneous nuclear ribonucleoproteins, splicing factors, translation factors, structural proteins, signal transduction proteins, chromatin associated proteins, transcription factors, proteasome subunits, mitochondrial proteins, nucleophosmin, SYT interacting protein SIP, PA1-G, CRHSP-24, HCD2, GMP synthase, FUSE binding protein 1, HDGF, PFC6D, KPF1, KNFE3 having the partial sequence TPGT (F/Mox)E, alpha NAC, ARDH, cargo selection protein, DAZ associated protein 1, DEAD box protein retinoblastoma, dihydrofolate reductase, hydroxyacyl-CoA dehydrogenase/3-ketoacyl-CoA thiolase/enoyl-CoA hydratase, ER-60, HCA56, Hsp-105, IGF-II. mRNA-binding protein 1, IGF-II mRNA-binding protein 3, lactate dehydrogenase A, NS-associated protein, RAD 21, RAD 23 homolog B, T-complex protein 1 beta subunit, thioredoxin like protein, an unnamed protein (NCBI 7020309) and c-Abl or a partial sequence derived therefrom by substitution and/or deletion of one or more amino acids.

23. Apoptosis-associated and/or -modified protein selected from the proteins as shown in Table 1, 2, 3, 4, 5, 6, 7 or 8 or proteolytic fragments thereof.

24. Use of a proteome of claim 20 or 21 or a protein of claims 22 or 23 as target for the diagnosis, prevention or treatment of apoptosis-associated diseases or in a method for identifying apoptosis modulators.

25. Method for inhibiting caspase cleavage of apoptosis-associated and/or modified proteins, characterized in that the caspase cleavage site is modified to avoid cleavage.

26. Use of a caspase cleavage site to design and/or screen for substances that inhibit or modulate caspase cleavage of proteins containing such cleavage sites.

27. Use according to claim 26, wherein the caspase cleavage site is contained in or combined with a reporter protein.

28. Use of a peptide or a protein containing a caspase cleavage site as a diagnostic tool to screen for caspased activity and/or to determine the effectivity of caspase cleavage inhibiting and/or modulating substances.

29. Method or use according to any one of claims 25-28, wherein the caspase cleavage site is characterized by the amino acid sequence XXXD, wherein X denotes any amino acid.

30. Method for use according to claim 29, wherein the caspase cleavage iste comprises one of the caspase sequences as shown in Table 9.
